SEQUENCE LISTING (40 sequences)

SEQ ID NO: 1 - DNA, 15089 bp, synthetic construct
  source 1..15089: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctaagcttcgaccgttttaacctcgaaatatgcacatgtaaggacggatgtgagcgaacgccagtgatgaccgggatcagaggtaacctaccatgcatggtggggattaggtgaccgttcgcaggtagtttgatcggagcgaatgttcggggggtctggcgtcagaggctctaaactttatgtaattcctgccgcgaaacacgcacgtatcaagcagtcagctgttctcttcgttcagcgcgcgccggtgttgcaaaacgagcgctcttcgccggcggtggctcgtgcgatagttcgttttgtcggtaatccgatgttgccgcgccgatatcatgtgatgttgtcacagtgcgcgaaattcgaatggtggtgtgcagtgattgtgttgtgacggcgagtggcgcgtgtgggtgcttagttttgggagatgttttcgtatttttttgttgataactcaggctttgttgctgtgttgtagtactattttccattgcgcggtgtccagcttttaattagtggcacatattcttagcaagtaaaaattattttgcatactattaaatttcttataaattattttctaaaattaagtttaccttttcaattttactaaaaatatcgatatatttattatcgctggaaaactacattattccacctctaagcaagaaccgttagttggcgcgtagctttaccacaaaattcctggaattgccgtacgcttcgcagttgtttcaagttgtctaagggacatacgattttttttgcctctgcgtcacgattttaacccaaaagcgagtttagttacatgtacattattattagataaagaagtatcgcgaatacttcagttgaataaactgtgcttggtttttgggtgaggatttgtggaaagtagagtgcgcgataaccgtaactttcgacccggattttcgccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcgaatccagctcttgagcagaggctctggcagcttttgcagcgtttatataacatgaaatatatatacgcattccgatcaaagctgggttaaccagatagatagtaacgtttaactagcgcctggcgcgttcgattttaaagagatttagagcgttatcccgtgcctatagatcttatagtatagacaacgaacgatcactcaaatccaagtcaataattcaagaatttatgtctgtttctgtgaaagggaaactaattttgttaaagaagacttacaatatcgtaatacttgttcaatcgtcgtggccgatagaaatatcttacaatccgaaagttgatgaatggaattggtctgcaactggtcgccttcattttgtaaaatgttcgcttgcggccgaaaaatttcgatatatctacaattgatatacaatctttactaaattttgaaaaaggaacactttgaatttcgaactgtcaatcgtatcattagaatttaatctaaatttaaatcttgctaaaggaaatagcaaggaacactttcgtcgtcggctacgcattcattgtaaaattttaaattttgacattccgcactttttgatagataagcgaagagtatttttattacatgtatcgcaagtattcatttcaacacacatatctatatatatatgttatatatttattcaattttgtttaccattgatcaatttttcacacatgaaacaaccgccagcattatataatttttttatttttttaaaaaatgtgtacacatattctgaaaatgaaaaattcaatggctcgagtgccaaataaagaaatggttacaatttaaggaaacaaatgtccttcttgcgtttgaaacaactaatccttttcgccctcgcggcgtttctcgaaaagggccaggaaggatccactagtgcgtatcgataagctttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatcgtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctaattgaattagctctaattgaattagtctctaattgaattagatccccgggcgagctcgaattaaccattgcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 2 - DNA, 13161 bp, Synthetic construct
  source 1..13161: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagcttgcaccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtttccagacttacaagccgaacacgacgttagaatcccaaaacaaactggttattgtggtaggtcatttgtttggcagaaagaaaaatcgagaaatttctctggccgttattcgttattctctcttttctttttgggtctctccctctctgcactaatgctctctcactctgtcacacagtgaacggcatactgctctcgttggttcgagagagcgcgcctcgaatgttcgcgaaaaacgtcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagatctgcggccgcggctaggcacaccgaaacgactaaccctaattcttatcctttacttcaggcggccgcggctcgagaatcaaaatgagctcgtatatggatcttgttgatgatgaaccagcgactttgtaccataagttcgtggagtgcttgaaagcgggcgagaacttctgcggagacaagctgagtggaattattaccatggcgatccttaaggcaatcaaggcgctgaccgaagttaaaaagacaacctttaacaaatataagacaacaatcaagcagggcctccagtatgacgtgggttcgtcgactatctcgtttgtgtatcacttgaaggactgtgatgagctgtccaggggcttgagcgatgccttcgagccctacaaattcaaaattaagtcgaataaagaggcaacctcgtttaagactctctttcgtggcccctcgtttggcagccagaagaactggcggaagaaagaggtggaccgcgaggtggataacttgtttcatagcaccgagacagacgaatcgattttcaaattcatcttgaacacgttggatagtattgagacacaaacgaacacggatcgccaaaagacggtgctgactttcatcctgttgatgacatttttcaactgctgtaggaacaatgacctgatgaacgttgatccctccacatttaagattgtgaaaaacaaattcgtcggatacctgctgcaggctgaggtcaaacagactaagacacgcaagtcgaggaacattttcttctttcccatccgcgagaatcgattcgatctgttcctggccttgcacgatttcttccgcacatgccagcctaccccaaagtcgcgtctttcggatcaagtatcggagcagaagtggcagcttttccgagattccatggtcattgattacaaccgtttctttaggaagtttccagcttcgcctattttcgcaataaagcacggccccaagtcccatctgggccggcatctgatgaacagctttctgcacaagaatgaactggattcctgggccaactccctgggcaactggagctcctcccagaatcaacgcgagtccggtgcgcgactgggctacacccacggtggtcgagatttgccacagccactgttcggattcctggctggctactgcgttcgaaatgaagagggccacatcgtgggcttgggtctggagaaggacattaatgatctgtttgacggtattatggacccacttaatgagaaggaggatacagagatttgtgaaagctacggcgagtgggccaaaattgtgagcaaggatgttctgatatttctgaagcgatatcattcgaagaacgcatgccggcgatatcagaactccacattgtacgcacgtacgttccttaagacagagtccgtcaccttgagcggctccaagggaagcgaagagccgagcagtcccgtcaggattccaatacttagtatgggaaaagcctccccaagcgagggccgaaagttgcgtgccagcgaacatgccaacgatgataatgagattgagaagatagattcggattcgtcgcaaagcgaagagatacctatcgaaatgtccgactccgaggacgaaacaacggcaagtaacattagcggaatttacctggacatgtcgaaagccaactccaacgtggtctattcccctccgagtcagactggccgtgccgccggtgccggccgtaaacgtggcgtgggtggtcgccgcaccgttgagtcgaagcggcggcgcgtcctggcaccaattaaccggtaagggtacctctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatctggccggccgtttaaacgaattcttgaagacgaaagggcctcgtgatacgcctatttcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 3 - DNA, 13669 bp, Synthetic construct
  source 1..13669: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcgcgcgaggtcgacggtatcgataagcttgatatcgaattcctgcagctggttgtaggtgcagttgcgcttcttgcacttggcgcacttgaggagatcggtcttggtgccctgcacggtggccagctgggcatcgttgatggcctccttgacgaacttctcgcgcagcttcttcatctcgtcgctggccatctcctccggcgtcattttggccagctgcttggcagtgacggcgccgcacataaagttgccgcgcaatccaggattcttggggtccttcagattggccacgcgtgacctaatgcgattcttgtacttcatatccgtgttattgaactcggagtaaatggcatcctccagttcggcggccatttcctccggctcaccgcatccctcgggcacttcgccaatcttcagtgcggtggccagcatttcgcggcatttgatgcggaccgcatctgtcatgccaccggagggaaacgaggtctgtgacgaggatgtcgagcctttcttctctttatccttggatgagctggatgaactggacttatccttgccggagattgacgacgacgacttggcggcactggtggacttcgaggcactgctgttgttggaggagccctccttagcggagctgttgttcggtgtggttggcgccgggctggccaggaagcgtttccagttcttgatcagcgtcttggccagagcgatcacctcgtcgtccttgctactcttgcgcagctcgtttacggtcatgccgatgcgcgttttggtcagaatgtcgagattgatgttaagcgtttgcagggccttcagcaggtccagagcctgatcctgtccctgtacggtggatatatgtatatttgtgacatgatgcgcggcacgttagctaatcccgatacttactgtgccgtcgctggccatcttgctcatcttcttttggattcgaaacacttcctcctccacgctcattttggcccacttaatttgatcagaggttaggttaactggcgtcggctgtcggctttaatcggtgctgcgggtctgcgacagttgtttatgcggaagccgggctatttctttttttccctccaataaatgttgtttactatttttttcgtttctgtattcgcagaacaagttgacagaacaccctaacgcgttttagtgttaccgggcggtggcttgtcaagaaataccttcccatggctaatacaattcccaactggaagtagtttgggtttcagaagtcaacagtgttaccgtattttaatgtacatctggcttttaagcgagtacacacacatagcattgcgatgttttttaaacttgcactgcttgcaagcaggggtccactgtatttcaatgaaagaacagcgtgttgggcgtttttgaattatactaattaccttcaataacaattccctatatatcacttagttttaataaataaatcgtttgagtgtggtctagagatgatttattatgatgaagtgcggttgttggggacaagcgggatacgtctgcgggtcaaatcggcggtgggacatagtgagagggcaacggcgaacaatcaccaacgcgccctggtacgcgagaaatgttgatatcgcacatgacctcagcatccaccttcagtttgagaacaaattaaatcaggcacataccagacaaccaggagaacactgaggaggcgtcatcaaagatatctttggacacgtggcataaacaagccaaactattatctaatcaatatttttatacaaattttgtatgtttccatgttatcttcgttgctcctgttagttactacgtttcatagcccttaaattggttgcttaacgtaaaataaaattatattgtataaaaataaagaggaattcacctataacaaacgaaaaagtaaataaataagaacttccccatgttaaaaatgccaccaccatctttacatataagtatgtacatatgaatgaatcacttaggttgcttgaatattacaatttcattaatagctaaatcacatttgatgtgttagtggaaaacggctatatatataaattatcgaaattgtgaatatcgaattgcgatagcacaatgggaaattccaccactagatttttggtacttttaacagatccttttcggttttgcgttgcgcgaagtgatctgaacttatcaaagtttgtaaggtaatacataaagtgaaaaagaattaatttgctcttgaaaggcaggccaaattaaaaaaaaaatatcaatatggacacgtatgccggtgcttacgaccgtcagagccgcgagcgggaaaattccagcgcagcaagtccagccacacagcgtagcgccaacgaggacaaggcggccgacttgcagcgagaagtcgagcgagatggtggcaggttcaggttcgtcgggcatttctccgaagctccaggcactagcgcgttcggaacagccgagcgcccggagttcgaacgcatcctgaacgagtgccgtgccggcaggctcaacatgatcatagtctatgacgtgagtcgcttctcgcgcctgaaggtcatggacgcgattccgattgtgagcgaattgctcgccctgggtgtgacgatagtgtccacgcaggaaggcgtgtttcggcagggaaacgtcatggacctgatacacctgattatgcgcttggacgcttcgcacaaggagtccagcctgaagagtgcgaagattctcgacaccaagaacctgcagcgcgaattgggcggttatgtcggcggtaaggctccctacggcttcgagcttgtttcggagacgaaggagatcacgcgtaacggccgaatggtcaatgtcgtgatcaacaagcttgcccactcgaccacaccccttacccgacccttcgagttcgagcccgacgttatccgttggtggtggcgtgagatcaagacccacaagcaccttcccttcaagccaggcagtcaagccgccattcacccgggcagcattacggggctgtgtaagcgcatggacgcagatgctgtgccaacaaggggtgagacgattggcaagaagaccgcttcaagcgcctgggaccccgcaaccgttatgcgaatccttcgtgacccgcgtattgcaggctttgccgctgaggtgatctacaagaagaagccggatggaacgccgaccacgaagatagagggttaccgcattcagcgcgatccgatcaccttgcgccccgtggagctcgattgcggacccatcatcgagcccgctgagtggtatgagctgcaggcgtggttggatggcaggggtcgcggcaagggtctttcccggggacaagccatactgagcgccatggacaagctgtactgcgaatgtggcgccgtgatgacttcgaagcgcggcgaggaatcgatcaaggactcctaccgctgccgtcgacggaaggtggttgacccaagcgcacctggtcagcatgagggcacgtgcaacgtgagcatggcggcactcgacaagttcgttgcggaacgcatcttcaacaagatcaggcacgctgaaggcgacgaagagacgttggcccttctgtgggaagccgcgcgacgcttcggcaagctcactgaggcgccagagaagtccggcgaacgggcgaaccttgttgcggagcgagccgatgccttgaacgccctcgaagagctgtacgaagaccgcgctgcaggagcttacgacggacccgttggcaggaagcacttccggaagcaacaggcagcgctgacactccggcagcaaggagcagaagagcgacttgccgagcttgaagccgccgaagccccaaagctccccttggaccaatggttccccgaagacgccgacgctgacccaactggaccgaagtcgtggtggggacgcgcatcagtggacgacaagcgcgtgtttgtgggcctcttcgtggacaagatcgttgtgaccaagagcactaccggcaggggacagggaacacccatcgagaagcgcgcttcgatcacgtgggcaaagcccccgaccgatgacgatgaagatgacgcccaggatggcaccgaagacgtagcggcctagctcgagaatgtatggacatagatttcaaataattaaatgtaatgcagtaattgatgtaattagttaaataagttagatattaataacatattaattatatgtattataacgcatataataataaaatgcatatttaggatatgcaagccattcgaattttctattttaatttccttttacaaagaaatgtataacaaaatataatttgaaaaaatgttctggctctaattcgatttcttttaagtattttgtgaactgcttttaataacgagcggttgcaaaacttaacagaaacgtgcactttgatcccaactaataccgtgtactatcacgtgtttggttttaagtactcatccttcgtgtttcgttgtttgttgtttgccttgctgtacacttggctcttgcgctctctcgctctccgttggagccggctttttgaatgcatgcctcgccctgctcgcgcccagtcctcctccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 4 - DNA, 14663 bp, Synthetic construct
  source 1..14663: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccattaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtacgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcgcgcgaggtcgacggtatcgataagcttgatatcgaattcctgcagctggttgtaggtgcagttgcgcttcttgcacttggcgcacttgaggagatcggtcttggtgccctgcacggtggccagctgggcatcgttgatggcctccttgacgaacttctcgcgcagcttcttcatctcgtcgctggccatctcctccggcgtcattttggccagctgcttggcagtgacggcgccgcacataaagttgccgcgcaatccaggattcttggggtccttcagattggccacgcgtgacctaatgcgattcttgtacttcatatccgtgttattgaactcggagtaaatggcatcctccagttcggcggccatttcctccggctcaccgcatccctcgggcacttcgccaatcttcagtgcggtggccagcatttcgcggcatttgatgcggaccgcatctgtcatgccaccggagggaaacgaggtctgtgacgaggatgtcgagcctttcttctctttatccttggatgagctggatgaactggacttatccttgccggagattgacgacgacgacttggcggcactggtggacttcgaggcactgctgttgttggaggagccctccttagcggagctgttgttcggtgtggttggcgccgggctggccaggaagcgtttccagttcttgatcagcgtcttggccagagcgatcacctcgtcgtccttgctactcttgcgcagctcgtttacggtcatgccgatgcgcgttttggtcagaatgtcgagattgatgttaagcgtttgcagggccttcagcaggtccagagcctgatcctgtccctgtacggtggatatatgtatatttgtgacatgatgcgcggcacgttagctaatcccgatacttactgtgccgtcgctggccatcttgctcatcttcttttggattcgaaacacttcctcctccacgctcattttggcccacttaatttgatcagaggttaggttaactggcgtcggctgtcggctttaatcggtgctgcgggtctgcgacagttgtttatgcggaagccgggctatttctttttttccctccaataaatgttgtttactatttttttcgtttctgtattcgcagaacaagttgacagaacaccctaacgcgttttagtgttaccgggcggtggcttgtcaagaaataccttcccatggctaatacaattcccaactggaagtagtttgggtttcagaagtcaacagtgttaccgtattttaatgtacatctggcttttaagcgagtacacacacatagcattgcgatgttttttaaacttgcactgcttgcaagcaggggtccactgtatttcaatgaaagaacagcgtgttgggcgtttttgaattatactaattaccttcaataacaattccctatatatcacttagttttaataaataaatcgtttgagtgtggtctagagatgatttattatgatgaagtgcggttgttggggacaagcgggatacgtctgcgggtcaaatcggcggtgggacatagtgagagggcaacggcgaacaatcaccaacgcgccctggtacgcgagaaatgttgatatcgcacatgacctcagcatccaccttcagtttgagaacaaattaaatcaggcacataccagacaaccaggagaacactgaggaggcgtcatcaaagatatctttggacacgtggcataaacaagccaaactattatctaatcaatatttttatacaaattttgtatgtttccatgttatcttcgttgctcctgttagttactacgtttcatagcccttaaattggttgcttaacgtaaaataaaattatattgtataaaaataaagaggaattcacctataacaaacgaaaaagtaaataaataagaacttccccatgttaaaaatgccaccaccatctttacatataagtatgtacatatgaatgaatcacttaggttgcttgaatattacaatttcattaatagctaaatcacatttgatgtgttagtggaaaacggctatatatataaattatcgaaattgtgaatatcgaattgcgatagcacaatgggaaattccaccactagatttttggtacttttaacagatccttttcggttttgcgttgcgcgaagtgatctgaacttatcaaagtttgtaaggtaatacataaagtgaaaaagaattaatttgctcttgaaaggcaggccaaattaaaaaaaaaatatcaatatggacacgtatgccggtgcttacgaccgtcagagccgcgagcgggaaaattccagcgcagcaagtccagccacacagcgtagcgccaacgaggacaaggcggccgacttgcagcgagaagtcgagcgagatggtggcaggttcaggttcgtcgggcatttctccgaagctccaggcactagcgcgttcggaacagccgagcgcccggagttcgaacgcatcctgaacgagtgccgtgccggcaggctcaacatgatcatagtctatgacgtgagtcgcttctcgcgcctgaaggtcatggacgcgattccgattgtgagcgaattgctcgccctgggtgtgacgatagtgtccacgcaggaaggcgtgtttcggcagggaaacgtcatggacctgatacacctgattatgcgcttggacgcttcgcacaaggagtccagcctgaagagtgcgaagattctcgacaccaagaacctgcagcgcgaattgggcggttatgtcggcggtaaggctccctacggcttcgagcttgtttcggagacgaaggagatcacgcgtaacggccgaatggtcaatgtcgtgatcaacaagcttgcccactcgaccacaccccttacccgacccttcgagttcgagcccgacgttatccgttggtggtggcgtgagatcaagacccacaagcaccttcccttcaagccaggcagtcaagccgccattcacccgggcagcattacggggctgtgtaagcgcatggacgcagatgctgtgccaacaaggggtgagacgattggcaagaagaccgcttcaagcgcctgggaccccgcaaccgttatgcgaatccttcgtgacccgcgtattgcaggctttgccgctgaggtgatctacaagaagaagccggatggaacgccgaccacgaagatagagggttaccgcattcagcgcgatccgatcaccttgcgccccgtggagctcgattgcggacccatcatcgagcccgctgagtggtatgagctgcaggcgtggttggatggcaggggtcgcggcaagggtctttcccggggacaagccatactgagcgccatggacaagctgtactgcgaatgtggcgccgtgatgacttcgaagcgcggcgaggaatcgatcaaggactcctaccgctgccgtcgacggaaggtggttgacccaagcgcacctggtcagcatgagggcacgtgcaacgtgagcatggcggcactcgacaagttcgttgcggaacgcatcttcaacaagatcaggcacgctgaaggcgacgaagagacgttggcccttctgtgggaagccgcgcgacgcttcggcaagctcactgaggcgccagagaagtccggcgaacgggcgaaccttgttgcggagcgagccgatgccttgaacgccctcgaagagctgtacgaagaccgcgctgcaggagcttacgacggacccgttggcaggaagcacttccggaagcaacaggcagcgctgacactccggcagcaaggagcagaagagcgacttgccgagcttgaagccgccgaagccccaaagctccccttggaccaatggttccccgaagacgccgacgctgacccaactggaccgaagtcgtggtggggacgcgcatcagtggacgacaagcgcgtgtttgtgggcctcttcgtggacaagatcgttgtgaccaagagcactaccggcaggggacagggaacacccatcgagaagcgcgcttcgatcacgtgggcaaagcccccgaccgatgacgatgaagatgacgcccaggatggcaccgaagacgtagcggcctagctcgagaatgtatggacatagatttcaaataattaaatgtaatgcagtaattgatgtaattagttaaataagttagatattaataacatattaattatatgtattataacgcatataataataaaatgcatatttaggatatgcaagccattcgaattttctattttaatttccttttacaaagaaatgtataacaaaatataatttgaaaaaatgttctggctctaattcgatttcttttaagtattttgtgaactgcttttaataacgagcggttgcaaaacttaacagaaacgtgcactttgatcccaactaataccgtgtactatcacgtgtttggttttaagtactcatccttcgtgtttcgttgtttgttgtttgccttgctgtacacttggctcttgcgctctctcgctctccgttggagccggctttttgaatgcatgcctcgccctgctcgcgcccagtcctcctccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 5 - DNA, 7559 bp, Synthetic construct
  source 1..7559: mol_type = other DNA
ggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagtcgtcttcgctttccgaattcgagctcggtacctcgcgaatgcatctagatatcagatctggatcccgggcccgtcgactgcagaggcctgcatgcaagcttgataacttgtgaagacgaccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacat

SEQ ID NO: 6 - DNA, 9690 bp, Synthetic construct
  source 1..9690: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctccgagctcggtacctcgcgaatgcatctagatgcaccattatgttccggaggcgcgccatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgaccggtggatcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcctaaatatagatcacggatccgatacgaatatgtatatgtataccacttatatatatatatatcagaatgctttgcatatatacggattttcggggcgttggtatttgttacgtaacgccaattttcatttagatatttgtgtacatttctccaggtttctttgtgaaaaaatctacagactattgatacgccaaaacaatgcttattattacaaatgtagagaggttaaactgtcaccacaactcactcaactggtaactactaccgtttggaacgcgatttcgatattttgaacaggtatctcgatatggtggcgtgttctcatacaagcaattcacgagatcgcaatgattcctttcagtaaacttcgaaggcggaacgattttcgacaatccacactaaatcgacaatcgaaaattcgattcgaattgacgcgaacttttaacgaatgcgttatttttaaaattctcgaaattatttcgaaactaaagtcacatacatattcgtctatatgtaattcgtaatttcgagaagctttttggctcatgctaccacatggcacacatagacataatgtataagtattggcgatctctttctaatgtctttggttatttttaaaacatattcgaaattatttcgcccatgtatatgagccgacaacgaattcgagtataatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 7 - DNA, 9868 bp, Synthetic construct
  source 1..9868: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagactagttgttcgccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccatgagcaagggcgaggagctgttcaccggcgtggtgcccatcctggtggagctggatggcgatgtgaacggccacaagttcagcgtgcgcggcgagggcgagggcgatgccaccatcggcaagctgaccctgaagttcatctgcaccaccggcaagctgcccgtgccctggcccaccctggtgaccaccctgacctacggcgtgcagtgcttcagccgctaccccgatcacatgaagcgccacgatttcttcaagagcgccatgcccgagggctacgtgcaggagcgcaccatcagcttcaaggatgatggcaagtacaagacccgcgccgtggtgaagttcgagggcgataccctggtgaaccgcatcgagctgaagggcaccgatttcaaggaggatggcaacatcctgggccacaagctggagtacaacttcaacagccacaacgtgtacatcaccgccgataagcagaagaacggcatcaaggccaacttcaccgtgcgccacaacgtggaggatggcagcgtgcagctggccgatcactaccagcagaacacccccatcggcgatggccccgtgctgctgcccgataaccactacctgagcacccagaccgtgctgagcaaggatcccaacgagaagtaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 8 - DNA, 10390 bp, Synthetic construct
  source 1..10390: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagactagttgttcgccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccatgaagatcgaggagggcaagttggtcatctggattaatggagataagggctataatggactggcagaggttggaaaaaaatttgagaaggatacaggcattaaggtcacagttgagcatccagataagttggaggagaagttcccgcaggtggctgcgacgggtgacggacccgacatcatattctgggctcatgaccgtttcggaggatacgcccagtccggcttgttggcggaaattacccctgataaagcttttcaggacaaactctaccccttcacctgggatgctgtccgatacaacggcaagctcattgcataccccattgcagttgaagcactgagtctgatttacaataaagacctgttgccgaatcctccgaagacctgggaggagattcccgctctggacaaggagctgaaggctaagggcaagtccgccctgatgttcaacctgcaagagccgtactttacttggcccctgatcgcagccgacggcggatacgccttcaaatacgagaacggaaagtacgatataaaggatgtcggagtcgacaacgccggcgctaaagcaggcctgactttcctggtggacctcataaagaacaagcatatgaatgccgacaccgattacagtatagcggaagccgcgttcaacaagggcgagactgccatgacaataaacggaccctgggcatggtccaacattgacacgagcaaggtgaactacggtgtaacggtgttgcctacgttcaagggacagccgagtaaaccattcgttggcgttttgtcggctggaattaatgctgctagtcccaataaggagttggctaaggagttcttggagaactacttgctgacagacgagggcttggaggcagtaaacaaggataagcccctgggtgcggttgccctcaagtcctatgaagaagagctcgtaaaagacccgcgtattgctgcgaccatggagaatgcccagaagggtgagattatgcccaatataccccagatgtccgcgttttggtatgcagttcgcaccgcggttataaacgcagcctcgggacggcagaccgtggatgaggacttgaaagacgcccagacacgcataaccaaacagtttcgcgaccatatggttctgcatgaatatgtcaacgcagcaggcattactagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 9 - DNA, 9454 bp, Synthetic construct
  source 1..9454: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctgtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgaggttgcttaagaataagtaattcttaagcaaccattacgccaagcttgcatgcaggcctctgcagtcgacgggcccgggatccgatcgacaagccgaacatatgggcgcgccgggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaatttcgaagatggtggagttgtcacagtcacacaagattcttcgctccaagatggttgttttatttacaaagtgaaatttattggagtcaattttccgtcggatggaccggttatgcaaaagaaaacaatgggttgggaagcatcgacggaacgtctgtatccacgagatggagttttgaaaggcgaaattcacaaagctcttaaactcaaagatggcggacattaccttgtagaatttaaaagcatttatatggctaagaaaccggtccaactccctggatattattatgtcgattcgaaactggatattacttcacataacgaagattatacgatagttgagcaatatgaacgcactgaaggtcgtcaccatctctttctctaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatggcgcgcctccggaacataatggtgcatctagatgcattcgcgaggtaccgagctcgaattcactggggttgcttaagaataagtaattcttaagcaacccaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 10 - DNA, 10418 bp, Synthetic construct
  source 1..10418: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctgtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgaggttgcttaagaataagtaattcttaagcaaccattacgccaagcttgcatgccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccattaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtacgatccaccggtcgccaccccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaatttcgaagatggtggagttgtcacagtcacacaagattcttcgctccaagatggttgttttatttacaaagtgaaatttattggagtcaattttccgtcggatggaccggttatgcaaaagaaaacaatgggttgggaagcatcgacggaacgtctgtatccacgagatggagttttgaaaggcgaaattcacaaagctcttaaactcaaagatggcggacattaccttgtagaatttaaaagcatttatatggctaagaaaccggtccaactccctggatattattatgtcgattcgaaactggatattacttcacataacgaagattatacgatagttgagcaatatgaacgcactgaaggtcgtcaccatctctttctctaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatggcgcgcctccggaacataatggtgcatctagatgcattcgcgaggtaccgagctcgaattcactggggttgcttaagaataagtaattcttaagcaacccaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 11 - DNA, 9622 bp, Synthetic construct
  source 1..9622: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagatactcgaattcgttgtcggctcatatacatgggcgaaataatttcgaatatgttttaaaaataaccaaagacattagaaagagatcgccaatacttatacattatgtctatgtgtgccatgtggtagcatgagccaaaaagcttctcgaaattacgaattacatatagacgaatatgtatgtgactttagtttcgaaataatttcgagaattttaaaaataacgcattcgttaaaagttcgcgtcaattcgaatcgaattttcgattgtcgatttagtgtggattgtcgaaaatcgttccgccttcgaagtttactgaaaggaatcattgcgatctcgtgaattgcttgtatgagaacacgccaccatatcgagatacctgttcaaaatatcgaaatcgcgttccaaacggtagtagttaccagttgagtgagttgtggtgacagtttaacctctctacatttgtaataataagcattgttttggcgtatcaatagtctgtagattttttcacaaagaaacctggagaaatgtacacaaatatctaaatgaaaattggcgttacgtaacaaataccaacgccccgaaaatccgtatatatgcaaagcattctgatatatatatatataagtggtatacatatacatattcgtatcggatccgtgatctatatttaggaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccacatgagcaagggcgaggagctgttcaccggcgtggtgcccatcctggtggagctggatggcgatgtgaacggccacaagttcagcgtgcgcggcgagggcgagggcgatgccaccatcggcaagctgaccctgaagttcatctgcaccaccggcaagctgcccgtgccctggcccaccctggtgaccaccctgacctacggcgtgcagtgcttcagccgctaccccgatcacatgaagcgccacgatttcttcaagagcgccatgcccgagggctacgtgcaggagcgcaccatcagcttcaaggatgatggcaagtacaagacccgcgccgtggtgaagttcgagggcgataccctggtgaaccgcatcgagctgaagggcaccgatttcaaggaggatggcaacatcctgggccacaagctggagtacaacttcaacagccacaacgtgtacatcaccgccgataagcagaagaacggcatcaaggccaacttcaccgtgcgccacaacgtggaggatggcagcgtgcagctggccgatcactaccagcagaacacccccatcggcgatggccccgtgctgctgcccgataaccactacctgagcacccagaccgtgctgagcaaggatcccaacgagaagtaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 12 - DNA, 8987 bp, Synthetic construct
  source 1..8987: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagcaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggatgagcaagggcgaggagctgttcaccggcgtggtgcccatcctggtggagctggatggcgatgtgaacggccacaagttcagcgtgcgcggcgagggcgagggcgatgccaccatcggcaagctgaccctgaagttcatctgcaccaccggcaagctgcccgtgccctggcccaccctggtgaccaccctgacctacggcgtgcagtgcttcagccgctaccccgatcacatgaagcgccacgatttcttcaagagcgccatgcccgagggctacgtgcaggagcgcaccatcagcttcaaggatgatggcaagtacaagacccgcgccgtggtgaagttcgagggcgataccctggtgaaccgcatcgagctgaagggcaccgatttcaaggaggatggcaacatcctgggccacaagctggagtacaacttcaacagccacaacgtgtacatcaccgccgataagcagaagaacggcatcaaggccaacttcaccgtgcgccacaacgtggaggatggcagcgtgcagctggccgatcactaccagcagaacacccccatcggcgatggccccgtgctgctgcccgataaccactacctgagcacccagaccgtgctgagcaaggatcccaacgagaagtaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 13 - DNA, 9512 bp, Synthetic construct
  source 1..9512: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagcaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggatgaaaattgaagagggcaagttggtcatctggattaatggagataagggctataatggactggcagaggttggaaaaaaatttgagaaggatacaggcattaaggtcacagttgagcatccagataagttggaggagaagttcccgcaggtggctgcgacgggtgacggacccgacatcatattctgggctcatgaccgtttcggaggatacgcccagtccggcttgttggcggaaattacccctgataaagcttttcaggacaaactctaccccttcacctgggatgctgtccgatacaacggcaagctcattgcataccccattgcagttgaagcactgagtctgatttacaataaagacctgttgccgaatcctccgaagacctgggaggagattcccgctctggacaaggagctgaaggctaagggcaagtccgccctgatgttcaacctgcaagagccgtactttacttggcccctgatcgcagccgacggcggatacgccttcaaatacgagaacggaaagtacgatataaaggatgtcggagtcgacaacgccggcgctaaagcaggcctgactttcctggtggacctcataaagaacaagcatatgaatgccgacaccgattacagtatagcggaagccgcgttcaacaagggcgagactgccatgacaataaacggaccctgggcatggtccaacattgacacgagcaaggtgaactacggtgtaacggtgttgcctacgttcaagggacagccgagtaaaccattcgttggcgttttgtcggctggaattaatgctgctagtcccaataaggagttggctaaggagttcttggagaactacttgctgacagacgagggcttggaggcagtaaacaaggataagcccctgggtgcggttgccctcaagtcctatgaagaagagctcgtaaaagacccgcgtattgctgcgaccatggagaatgcccagaagggtgagattatgcccaatataccccagatgtccgcgttttggtatgcagttcgcaccgcggttataaacgcagcctcgggacggcagaccgtggatgaggacttgaaagacgcccagacacgcataaccaaacagtttcgcgaccatatggttctgcatgaatatgtcaacgcagcaggcattacttaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 14 - DNA, 10147 bp, Synthetic construct
  source 1..10147: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagatactcgaattcgttgtcggctcatatacatgggcgaaataatttcgaatatgttttaaaaataaccaaagacattagaaagagatcgccaatacttatacattatgtctatgtgtgccatgtggtagcatgagccaaaaagcttctcgaaattacgaattacatatagacgaatatgtatgtgactttagtttcgaaataatttcgagaattttaaaaataacgcattcgttaaaagttcgcgtcaattcgaatcgaattttcgattgtcgatttagtgtggattgtcgaaaatcgttccgccttcgaagtttactgaaaggaatcattgcgatctcgtgaattgcttgtatgagaacacgccaccatatcgagatacctgttcaaaatatcgaaatcgcgttccaaacggtagtagttaccagttgagtgagttgtggtgacagtttaacctctctacatttgtaataataagcattgttttggcgtatcaatagtctgtagattttttcacaaagaaacctggagaaatgtacacaaatatctaaatgaaaattggcgttacgtaacaaataccaacgccccgaaaatccgtatatatgcaaagcattctgatatatatatatataagtggtatacatatacatattcgtatcggatccgtgatctatatttaggaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccacatgaagatcgaggagggcaagttggtcatctggattaatggagataagggctataatggactggcagaggttggaaaaaaatttgagaaggatacaggcattaaggtcacagttgagcatccagataagttggaggagaagttcccgcaggtggctgcgacgggtgacggacccgacatcatattctgggctcatgaccgtttcggaggatacgcccagtccggcttgttggcggaaattacccctgataaagcttttcaggacaaactctaccccttcacctgggatgctgtccgatacaacggcaagctcattgcataccccattgcagttgaagcactgagtctgatttacaataaagacctgttgccgaatcctccgaagacctgggaggagattcccgctctggacaaggagctgaaggctaagggcaagtccgccctgatgttcaacctgcaagagccgtactttacttggcccctgatcgcagccgacggcggatacgccttcaaatacgagaacggaaagtacgatataaaggatgtcggagtcgacaacgccggcgctaaagcaggcctgactttcctggtggacctcataaagaacaagcatatgaatgccgacaccgattacagtatagcggaagccgcgttcaacaagggcgagactgccatgacaataaacggaccctgggcatggtccaacattgacacgagcaaggtgaactacggtgtaacggtgttgcctacgttcaagggacagccgagtaaaccattcgttggcgttttgtcggctggaattaatgctgctagtcccaataaggagttggctaaggagttcttggagaactacttgctgacagacgagggcttggaggcagtaaacaaggataagcccctgggtgcggttgccctcaagtcctatgaagaagagctcgtaaaagacccgcgtattgctgcgaccatggagaatgcccagaagggtgagattatgcccaatataccccagatgtccgcgttttggtatgcagttcgcaccgcggttataaacgcagcctcgggacggcagaccgtggatgaggacttgaaagacgcccagacacgcataaccaaacagtttcgcgaccatatggttctgcatgaatatgtcaacgcagcaggcattacttaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 15 - DNA, 10496 bp, Synthetic construct
  source 1..10496: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctccgagctcggtacctcgcgaatgcatctagatgcaccattatgttccggaggcgcgccatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgaccggtggatcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcctaaatatagatcacggatccgatacgaatatgtatatgtataccacttatatatatatatatcagaatgctttgcatatatacggattttcggggcgttggtatttgttacgtaacgccaattttcatttagatatttgtgtacatttctccaggtttctttgtgaaaaaatctacagactattgatacgccaaaacaatgcttattattacaaatgtagagaggttaaactgtcaccacaactcactcaactggtaactactaccgtttggaacgcgatttcgatattttgaacaggtatctcgatatggtggcgtgttctcatacaagcaattcacgagatcgcaatgattcctttcagtaaacttcgaaggcggaacgattttcgacaatccacactaaatcgacaatcgaaaattcgattcgaattgacgcgaacttttaacgaatgcgttatttttaaaattctcgaaattatttcgaaactaaagtcacatacatattcgtctatatgtaattcgtaatttcgagaagctttttggctcatgctaccacatggcacacatagacataatgtataagtattggcgatctctttctaatgtctttggttatttttaaaacatattcgaaattatttcgcccatgtatatgagccgacaacgaattcgagtataatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaatcgaagttcacccggatatctttcctatatatactgtactcttgcagaatatgtacttgggaatcataaggatgcgaaggacgtacgcgctgtgtctgtaactaaggggttgctttcgggtacctaccatgccaatgatgggactaccgccggataggaatcccttttgggtgcacaccgtcacctccagttccgtctgcctgagttcggcaatggaaatcaggtactcaaaggatgcatcgtagacggggttgcagttgtccttgatcacgctcgtcttgcgtttcgactccttggtgcgtccaggcaacagatacagcttaacatacggatcggggatattgctgggatcgcgaagtggtatcttctggattttgtgtatggtcacgtctagtttttgacgctgggcgctgtagcggatcgacaactgcattcgtccaaggccatgttcgccgggtgaggaggtggagtctggaaagcggtgggtcagcacagaggtggccagctcgctgatgggtttttcgctggaggtggaggccgacatggccggggagattttcgtagccgccacaggatcttcttcactgatgggcgattcggcggacaaacgcttgctggatgcctgagattcctaaaagcaatggcaaattagattcaattaaaacagaaattgtttatataacagagtaaaagcaaaaaaaaaggaatcacagattctccccgacggggaattgaaccccgttctcccgcgtgacaggcggggatactaaccactatactatcgaggacgttgaaaatgaattcaattaagacgtcagaattctcgaggcggccgcatgtgagtctccctatagtgagtcgtattaatttcgcgggcgcgtcagcgggagagccgttgtaaggcggca

SEQ ID NO: 16 - DNA, 11224 bp, Synthetic construct
  source 1..11224: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctgtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgaggttgcttaagaataagtaattcttaagcaaccattacgccaagcttgcatgccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccattaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtacgatccaccggtcgccaccccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaatttcgaagatggtggagttgtcacagtcacacaagattcttcgctccaagatggttgttttatttacaaagtgaaatttattggagtcaattttccgtcggatggaccggttatgcaaaagaaaacaatgggttgggaagcatcgacggaacgtctgtatccacgagatggagttttgaaaggcgaaattcacaaagctcttaaactcaaagatggcggacattaccttgtagaatttaaaagcatttatatggctaagaaaccggtccaactccctggatattattatgtcgattcgaaactggatattacttcacataacgaagattatacgatagttgagcaatatgaacgcactgaaggtcgtcaccatctctttctctaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatggcgcgcctccggaacataatggtgcatctagatgcattcgcgaggtaccgagctcgaattcactggggttgcttaagaataagtaattcttaagcaacccaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaatcgaagttcacccggatatctttcctatatatactgtactcttgcagaatatgtacttgggaatcataaggatgcgaaggacgtacgcgctgtgtctgtaactaaggggttgctttcgggtacctaccatgccaatgatgggactaccgccggataggaatcccttttgggtgcacaccgtcacctccagttccgtctgcctgagttcggcaatggaaatcaggtactcaaaggatgcatcgtagacggggttgcagttgtccttgatcacgctcgtcttgcgtttcgactccttggtgcgtccaggcaacagatacagcttaacatacggatcggggatattgctgggatcgcgaagtggtatcttctggattttgtgtatggtcacgtctagtttttgacgctgggcgctgtagcggatcgacaactgcattcgtccaaggccatgttcgccgggtgaggaggtggagtctggaaagcggtgggtcagcacagaggtggccagctcgctgatgggtttttcgctggaggtggaggccgacatggccggggagattttcgtagccgccacaggatcttcttcactgatgggcgattcggcggacaaacgcttgctggatgcctgagattcctaaaagcaatggcaaattagattcaattaaaacagaaattgtttatataacagagtaaaagcaaaaaaaaaggaatcacagattctccccgacggggaattgaaccccgttctcccgcgtgacaggcggggatactaaccactatactatcgaggacgttgaaaatgaattcaattaagacgtcagaattctcgaggcggccgcatgtgagtctccctatagtgagtcgtattaatttcgcgggcgcgtcagcgggagagccgttgtaaggcggca

SEQ ID NO: 17 - DNA, 8792 bp, Synthetic construct
  source 1..8792: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagtcgtcttcgctttccgaattcgagctcggtacctcgcgaatgcatctagatatcagatctggatcccgggcccgtcgactgcagaggcctgcatgcaagcttgataacttgtgaagacgaccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaatcgaagttcacccggatatctttcctatatatactgtactcttgcagaatatgtacttgggaatcataaggatgcgaaggacgtacgcgctgtgtctgtaactaaggggttgctttcgggtacctaccatgccaatgatgggactaccgccggataggaatcccttttgggtgcacaccgtcacctccagttccgtctgcctgagttcggcaatggaaatcaggtactcaaaggatgcatcgtagacggggttgcagttgtccttgatcacgctcgtcttgcgtttcgactccttggtgcgtccaggcaacagatacagcttaacatacggatcggggatattgctgggatcgcgaagtggtatcttctggattttgtgtatggtcacgtctagtttttgacgctgggcgctgtagcggatcgacaactgcattcgtccaaggccatgttcgccgggtgaggaggtggagtctggaaagcggtgggtcagcacagaggtggccagctcgctgatgggtttttcgctggaggtggaggccgacatggccggggagattttcgtagccgccacaggatcttcttcactgatgggcgattcggcggacaaacgcttgctggatgcctgagattcctaaaagcaatggcaaattagattcaattaaaacagaaattgtttatataacagagtaaaagcaaaaaaaaaggaatcacagattctccccgacggggaattgaaccccgttctcccgcgtgacaggcggggatactaaccactatactatcgaggacgttgaaaatgaattcaattaagacgtcagaattctcgaggcggccgcatgtgagtctccctatagtgagtcgtattaatttcgcgggcgcgtcagcgggagagccgttgtaaggcggca

SEQ ID NO: 18 - DNA, 8792 bp, Synthetic construct; identical to SEQ ID NO: 17
  source 1..8792: mol_type = other DNA

SEQ ID NO: 19 - DNA, 10998 bp, Synthetic construct
  source 1..10998: mol_type = other DNA
tgccgccttacaacggctctcccgctgacgcgcccgcgaaattaatacgactcactatagggagactcacatgcggccgcctcgagaattctgacgtcttaattgaattcattttcaacgtcctcgatagtatagtggttagtatccccgcctgtcacgcgggagaacggggttcaattccccgtcggggagaatctgtgattcctttttttttgcttttactctgttatataaacaatttctgttttaattgaatctaatttgccattgcttttaggaatctcaggcatccagcaagcgtttgtccgccgaatcgcccatcagtgaagaagatcctgtggcggctacgaaaatctccccggccatgtcggcctccacctccagcgaaaaacccatcagcgagctggccacctctgtgctgacccaccgctttccagactccacctcctcacccggcgaacatggccttggacgaatgcagttgtcgatccgctacagcgcccagcgtcaaaaactagacgtgaccatacacaaaatccagaagataccacttcgcgatcccagcaatatccccgatccgtatgttaagctgtatctgttgcctggacgcaccaaggagtcgaaacgcaagacgagcgtgatcaaggacaactgcaaccccgtctacgatgcatcctttgagtacctgatttccattgccgaactcaggcagacggaactggaggtgacggtgtgcacccaaaagggattcctatccggcggtagtcccatcattggcatggtaggtacccgaaagcaaccccttagttacagacacagcgcgtacgtccttcgcatccttatgattcccaagtacatattctgcaagagtacagtatatataggaaagatatccgggtgaacttcgattaagaagtctacaggaatgggcatctagatgcaccattatgttccggaggcaactttgtattaaaaagttgaacgagaaacgtaaaatgatataaatatcaatatattaaattagattttgcataaaaaacagactacataatactgtaaaacacaacatatccagtcactatgcgacgtgcagactggctgtgtataagggagcctgacatttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgccacgcgtttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggcccatagtgactggatatgttgtgttttacagtattatgtagtctgttttttatgcaaaatctaatttaatatattgatatttatatcattttacgtttctcgttcaacttttgtatacaaagttgattatactcgaattcgttgtcggctcatatacatgggcgaaataatttcgaatatgttttaaaaataaccaaagacattagaaagagatcgccaatacttatacattatgtctatgtgtgccatgtggtagcatgagccaaaaagcttctcgaaattacgaattacatatagacgaatatgtatgtgactttagtttcgaaataatttcgagaattttaaaaataacgcattcgttaaaagttcgcgtcaattcgaatcgaattttcgattgtcgatttagtgtggattgtcgaaaatcgttccgccttcgaagtttactgaaaggaatcattgcgatctcgtgaattgcttgtatgagaacacgccaccatatcgagatacctgttcaaaatatcgaaatcgcgttccaaacggtagtagttaccagttgagtgagttgtggtgacagtttaacctctctacatttgtaataataagcattgttttggcgtatcaatagtctgtagattttttcacaaagaaacctggagaaatgtacacaaatatctaaatgaaaattggcgttacgtaacaaataccaacgccccgaaaatccgtatatatgcaaagcattctgatatatatatatataagtggtatacatatacatattcgtatcggatccgtgatctatatttaggaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggccgcggctcgagggtaccaatcaaacatgtccaaaggtgaagaactgtttaccggagtagtcccgatattggttgaactcgacggcgatgtcaacggtcataaattcagtgtgtccggcgagggtgagggcgacgccacatacggtaagctgacgttgaagttcatatgcaccacgggcaagctgcccgtgccatggccgacgttggtcacgacgctgacgtatggtgtccagtgtttcagccgttaccccgatcatatgaagcagcatgactttttcaagtcggcgatgccggagggatacgttcaagagaggaccattttcttcaaggatgacggcaactataagacgcgagcggaggtgaaatttgaaggcgacacactcgttaaccgtattgagttgaagggcattgattttaaggaggatggcaacattctgggccataagttggaatacaactacaactcgcataatgtgtatataatggcagataagcagaaaaacggaataaaggttaacttcaagattcgccacaacatagaggacggttccgtgcaacttgcagatcattaccaacagaacacacccattggagatggcccagttctcttgccagacaatcactacctttccacacagtccgcgttgagcaaggaccccaatgaaaagcgggaccacatggtgttgctggagtttgtgaccgcagctggtattacacacggcatggatgagctctacaagtaatctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatggcgcgcctccggaacataatggtgcatctagatgcattcgcgaggtaccgagctcggagctggacaactttgtatagaaaagttgaacgagaaacgtaaaatgatataaatatcaatatattaaattagattttgcataaaaaacagactacataatactgtaaaacacaacatatccagtcactatgcgacgtgcagactggctgtgtataagggagcctgacattatattccccagaacatcaggttaatggcgtttttgatgtcattttcgcggtggctgagatcagccacttcttccccgataacggaaaccggcacactggccatatcggtggtcatcatgcgccagctttcatccccgatatgcaccaccgggtaaagttcacgggagactttatctgagagcaagcgcgcagacgcaagtggaataaccatgcgacggcccggtgtatcgataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccattaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgtagctcctgaaaatctcgacggatcctaactcaaaatccacacattatacgagccggaagcataaagtgtaaagcctggggtgcctaatgcggcccatagtgactggatatgttgtgttttacagtattatgtagtctgttttttatgcaaaatctaatttaatatattgatatttatatcattttacgtttctcgttcaactttattatacaaagttgaggccccgacgttaaattgaaaataggtctatatatacgaactgagtctggaaaaagaagttgagaattataaaaagtagtgagcactggcgccctctctgcttggcgagctaaccttttcgcctcttggctgagtaggtggcgtttcattctactctgtaaaattaatgtagaattgaaacactatggtcaaaaaatacttaggggcataagttataaaacgtatgaaatgaatttttatcaacctgggctattcaaaaattttcgaattattttatgtattttttttaatcgtttttcattataggttaaaatacactttaaaaggaattctttcctgtaaaataaatataaataaatatgctttattgacagaaaatttgatgtttttgtatttgagtaggagcaatcacaggtgagcaaaaaacccatatgttcggcttgtcgatcggatcccctccgtaccaagacttcgaattcggatccctcgagcgatacacacttctatagtgtcacctaaatgcgtttaaaccttcctgcaggtgacgatttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtc

SEQ ID NO: 20 - DNA, 13948 bp, Synthetic construct
  source 1..13948: mol_type = other DNA
actttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagtcgtcttcgctttccgaattcgagctcggtacctcgcgaatgcatctagatatcagatctggatcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtacgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcgcgccaagcttggtaccgtctgccctttattggacttttccgtcctgcagccaatccgccgcaccctcagctccaaagcacgcatatgacccgtgagcaggcagagaaacgcaggcggataggagtcactagcgcagttctgtatagcctgcactatcaagccgaagagctggtagatattgatgagcacatagtttctggtggagtgcatccagtcaacgccgaaccaggccggatacaggagctctcgatccgggcgaacgaccacgcggacgcaactcaaagtagtgccaaatacgaaaatactggcaaaggtgcggaaagtgccctgtgcgatattcacttccttcctcaaggcagaaatctcctcgggatcgcccaccagccgggctctagcgtccatgagccttaggagagagcgaatctcatggagctgcttcctcaccatgtacacattcgcaaacttcaaattggccacaatatcggtaatagttatggtcaggttctcgcacaatccggccatgttggtggtgaacagcagcctggccagcaagctcaagggaaacagcacggtgaccaccaagttgaccgtaatggagtataccacgtaaagcaggcttgaaacgcccggaggacgcatcaggccagtgagctcccaaacgcgccagtggtagtacacagcactgtgggtgttcagtttgaaatcctcttgcttctccattgtctcggttacctttgggcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatattccccattgataataataattccactatatgttcaattgggtttcaccgcgcttagttacatgacgagccctaacgagccgtcggtggtctataaactgtgccttacaaatacttgtaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttaactggttgggagattggtgctatactagagtttttacgttgggtcccctaggctctactattccgctaagtggggctgctttggcgttacttgtcaggtggatccggtcatgaagatcaagagcgtgattaatctacaagtcggtaaagagaacagcggaattgggtcagggacggggttgggtggggaaaaagaacacaaaaacaaatccgtagatgcacaccatcgagcacacgttcacagagtgatcttaatttgtagtttttaagaattaatttaaaaatacaatcaagtggtgtaatctaaggcctcgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttgaaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttaacttatacggttcttgtagtaagttttttgccaaagggattgaggtgaaccaattgtcacacgtaatattacgacaactaccgtgcacaggctttgataactccttcacgtagtattcaccgagtggtactccgttggtctgtgttcctcttcccaaataaggcattccatttatcatatacttcgtaccactgtcacacatcatgaggatttttattccatacttacttggcttgtttgggatatacatcctaaacggacaccgtcctctaaaaccaagtaactgttcatctatggtcaaatgagcccctggagtgtaattttgtatgcactgatggataaagagatcccatatttttctaacaggagtaaatacatcgttttctcgaagtgtgggccgtatacttttgtcatccattctaagacatcgtatcaaaaaatcaaaacgatcacgactcattacagagacgtacaccattgacaaagatcgatcaaagaggtcatctgtggacatgtggttatcttttctcactgctgtcattaccagaataccaaagaaagcatagatttcatcttcattcgtgtcacgaaatgtagcacctgtcatagattcccgacgtttcaatgatatctcagcatttgtccattttacaatttccgaaattatctcatcagtaaaaaatagtttgaagcataaaagtgggtcatatatattgcggcacatacgcgtcggacctctttgagatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccaggatcccacgctgtggatgaggaggaggggaaaacggatgattagttacacaccccaagaaaatcgataattgcagagccgcgcccaacttgagccggcactgcgtgcgagtgagagaggtagctaaatggtatactgatttttttgctctactttgccgacgccggaattttactctctcgccattttctctttaattctatccaaaatctacagttatgcgtcgaagtatcatggtaaccgaatttagaatttttaagattaatgaagcgttccagcagcatagaaataatgaatattttttttatctagattttaagagccattcctcattgcgaacaaaaaaaaaaacaggcatgacgtcatcacttttgaggaaaaaacgcaggccggccgccattttgagaaaaagccgcatcgcgtttttgcccattgaattttgcccacacaaatttcttttcagcattatttcactagatttttttttaaagtactcaccatattgagtttttattggaagtgtttcacacgcgacaaggaaaattcacagaattgtgtagagctacgatttcgaggtctgactcagagagcgtatacgttcgaactgccggctatgaggcttgacaaacgtaaaacgaatatgaaatgtggaaagcgggctgctttatataggcaccggagctagagacggcaagctgtcgacttaaacgccgcataaccgataactgaagtgggagaggtaatggcacttggtctctctaaaacttgctgcggtattttgcaggactagttgggactcgaagcggccgccatatgcatcctaggcctattaatattccggagtatacgtagccggctaacgttaacaaccggtacctctagaactatagctagcatgcaagcttgataacttgtgaagacgaccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggcag

SEQ ID NO: 21 - DNA, 15316 bp, Synthetic construct
  source 1..15316: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctccgagctcggtacctcgcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccagggccgcaagcttgcatgcctgcaggtcggagtactgtcctccgagcggagtactgtcctccgagcggagtactgtcctccgagcggagtactgtcctccgagcggagtactgtcctccgagcggagactctagccctagggcatgcctgcaggtcggagtactgtcctccgagcggagtactgtcctccgagcggagtactgtcctccgagcggagtactgtcctccgagcggagtactgtcctccgagcggagactctagcgctagcgacgtcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagatctaaaaggtaggttcaaccactgatgcctaggcacaccgaaacgactaaccctaattcttatcctttacttcaggcggccgcggctcgagggtaccaatcaaaatgcccaaaaagaaacgcaaggtggacaagaaatactccatcggactggacatcgggacgaactcagtgggttgggccgtgatcaccgatgaatacaaggtgcccagcaaaaagttcaaggtgctgggcaacaccgaccgccactccatcaaaaagaacctcattggcgctctcctgttcgacagcggcgaaactgctgaggccacacgcctgaagcgcaccgcacgcagaaggtacaccaggagaaaaaaccgcatctgctatctgcaggaaatcttttccaatgagatggctaaggtggatgacagctttttccaccgcctcgaagagtcttttctggtggaagaggacaaaaagcacgagcgccaccctatctttggtaacattgtggacgaggtggcgtatcacgagaagtatcctaccatttaccaccttcgcaaaaagctggtcgatagcactgataaggccgatctgcgcctcatctacctggccttggcccatatgatcaagttccgtgggcacttcctcatcgaaggtgatctgaaccctgacaatagcgacgtggataagctgttcatccagctggtccagacttacaaccagctgttcgaggaaaaccccatcaatgcttccggggtagatgctaaggctatcctgagtgctaggctgtctaagtctcggcgtctggagaatctgattgcgcagctgcctggtgagaaaaagaatggactgttcggcaacctgatcgccctgtccctgggccttaccccgaacttcaagtctaactttgacctcgctgaggacgccaaacttcagctgagcaaggacacatacgacgatgaccttgacaacctgctcgcccagatcggtgaccagtacgccgatctgttcctggcagccaagaacctgtctgacgcaatcttgctcagcgatatcctgcgcgtcaacaccgagatcaccaaggcacctctgagcgcaagcatgattaaacgctatgacgaacatcaccaggacctcacgctcctgaaagccctggtccgccaacagctgcccgagaaatacaaggagattttctttgatcagtccaagaacgggtacgctggctatatcgatggcggagcgagccaggaagagttctacaagtttatcaagccgatcctggagaaaatggacggcaccgaagagctcctggtgaagctgaacagggaggaccttctgcgcaaacagcggacctttgataatgggagcatcccccaccagattcatttgggtgagctgcacgccatcctgcgccgtcaagaggacttctaccccttcctcaaagataaccgcgaaaagattgaaaagattctgacctttcgcatcccatattacgtgggccccctcgccagaggcaacagccgctttgcatggatgacgcgcaagtcagaagagaccattaccccctggaactttgaagaggtcgttgacaaaggtgcatcagctcagagctttatcgagcggatgactaacttcgataagaacctgccaaacgagaaggtactgcctaagcattctctcctgtacgaatactttaccgtgtataatgagctgacgaaggtgaaatacgtcactgagggtatgcgcaagcctgcgttccttagcggagagcagaaaaaggctattgtggatctcttgtttaagacaaacaggaaggttaccgtgaagcaactgaaggaggattatttcaaaaagattgagtgtttcgactctgtggaaatctccggcgtcgaggaccggttcaacgcatctttgggcacgtatcatgacctcctgaagattatcaaggacaaagactttctggataatgaagagaacgaagatatcctcgaggacatcgtgctgactctcactctgtttgaggaccgcgaaatgatcgaagagcgcctcaagacttatgcccaccttttcgatgacaaagtgatgaagcagctcaaacgtcggcgttataccggatggggacgcctgtctcgtaagctgatcaacggcatccgggacaagcaatccggcaagaccattctggacttccttaagagcgatggtttcgccaatagaaatttcatgcaactcatccacgatgacagtcttacattcaaggaggacatccagaaggcccaggtgagcggacagggcgacagccttcatgagcacatcgctaacctggcaggatcccccgccatcaaaaagggcatcctgcagaccgtcaaggttgtggacgagttggtgaaagtgatgggccgccacaagcctgagaatatcgtgattgagatggcgcgtgaaaaccagactacccaaaagggacagaagaacagtcgcgaacgcatgaagcgcatcgaagagggtattaaggagctgggtagtcagattctgaaggagcaccctgtggagaacacccagctccagaacgagaaactctacctgtactatctgcagaacggacgcgatatgtacgtggaccaggagctcgacatcaacaggcttagcgactacgacgtagatcatatcgtgcctcagtctttcctgaaagatgactctatcgacaacaaggtgctgacacgctctgataagaatcgtggcaagtccgacaacgttccttctgaagaggtggtcaagaaaatgaagaattactggaggcagctcctgaacgctaaactgatcactcaaagaaaattcgacaacctgaccaaggctgaacgcggtggcctgagcgagctggataaagcgggtttcatcaagagacagctggtcgaaacgcgtcaaatcacaaagcacgtagctcagatcctggattccaggatgaacaccaaatacgacgaaaacgataagctgattcgcgaggtgaaggtgattacccttaagtctaagctggtgagcgattttcgcaaagactttcagttttataaagtccgtgagatcaacaattatcatcacgctcatgatgcctatctgaatgctgtggtcggaactgctctgattaaaaagtaccctaagctcgagagcgaattcgtatatggagactacaaagtgtatgatgtgaggaagatgatcgccaagtccgagcaggaaatcggaaaggcgaccgccaagtactttttctatagcaacatcatgaattttttcaaaaccgagattaccttggctaacggtgagatccgcaagcgtcccctcatcgaaacgaacggcgaaacgggtgaaatcgtttgggataaaggtcgggacttcgccacagtgcgcaaggtgctctcaatgccacaggtgaacatcgtgaaaaagaccgaggtgcaaactggcggtttcagcaaagagtccatcctcccgaaacgcaactccgacaagctgattgcccggaaaaaggattgggatcccaaaaagtatggtggcttcgactctccaacagtggcttattctgtgttggttgtcgcgaaggtggagaagggcaaaagtaagaaactgaagtccgtgaaggagctcctgggtatcaccatcatggagagatcttccttcgagaagaaccccatcgatttcctcgaggccaagggctataaggaggtgaaaaaggatttgatcattaagcttcccaagtacagcctgttcgagctggagaacggccgcaagcgcatgctcgctagtgctggtgagctgcagaagggaaacgagctggcactcccaagtaagtatgtgaactttctgtatctggctagccattatgagaagctcaaaggtagcccagaggacaatgagcagaaacagctttttgtggaacagcacaagcactatttggacgaaattatcgagcagatcagcgagtttagcaagagggtaatcctggccgatgctaacctggacaaggtcctgagtgcttataacaagcaccgcgacaagccgatccgtgaacaggccgagaatattatccacctcttcactcttacgaatctgggcgctccagctgccttcaagtacttcgatacgactattgacagaaaaaggtatacctccacgaaggaagtgctggatgccaccctgatccaccaatccatcactggtttgtatgaaacgcgcatcgacctgtcacccaagaaaaagcgcaaggtgcagctgggcggggactagtctagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccggccgtttaaacgaattcttcgaatgcatctagatgcaccattatgttccggaggcgcgccatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgaccggtggatcccgggcccgcggtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcctaaatatagatcacggatccgatacgaatatgtatatgtataccacttatatatatatatatcagaatgctttgcatatatacggattttcggggcgttggtatttgttacgtaacgccaattttcatttagatatttgtgtacatttctccaggtttctttgtgaaaaaatctacagactattgatacgccaaaacaatgcttattattacaaatgtagagaggttaaactgtcaccacaactcactcaactggtaactactaccgtttggaacgcgatttcgatattttgaacaggtatctcgatatggtggcgtgttctcatacaagcaattcacgagatcgcaatgattcctttcagtaaacttcgaaggcggaacgattttcgacaatccacactaaatcgacaatcgaaaattcgattcgaattgacgcgaacttttaacgaatgcgttatttttaaaattctcgaaattatttcgaaactaaagtcacatacatattcgtctatatgtaattcgtaatttcgagaagctttttggctcatgctaccacatggcacacatagacataatgtataagtattggcgatctctttctaatgtctttggttatttttaaaacatattcgaaattatttcgcccatgtatatgagccgacaacgaattcgagtataatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 22 - DNA, 13856 bp, Synthetic construct
  source 1..13856: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggaggctcgcgtatcggtgattcattctgctaaccagtaaggcaaccccgccagcctagccgggtcctcaacgacaggagcacgatcatgcgcacccgtggccagggccgcaagcttgcatgcctgcaggtcggagtactgtcctccgagcggagtactgtcctccgagcggagtactgtcctccgagcggagtactgtcctccgagcggagtactgtcctccgagcggagactctagccctagggcatgcctgcaggtcggagtactgtcctccgagcggagtactgtcctccgagcggagtactgtcctccgagcggagtactgtcctccgagcggagtactgtcctccgagcggagactctagcgctagcgcatgcctgcaggtcggagtactgtcctccgagcggagtactgtcctccgagcggagtactgtcctccgagcggagtactgtcctccgagcggagtactgtcctccgagcggagactctagcactagtgcatgcctgcaggtcggagtactgtcctccgagcggagtactgtcctccgagcggagtactgtcctccgagcggagtactgtcctccgagcggagtactgtcctccgagcggagactctagcgacgtcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagatctaaaaggtaggttcaaccactgatgcctaggcacaccgaaacgactaaccctaattcttatcctttacttcaggcggccgcaacttaaaaaaaaaaatcaaaatgggctcacatcatcaccaccaccacggaatggctagcatgacgggcggacaacagatgggccgcgatctgtacgatgatgatgataaggacctggcgacaatggttgacagcagccggcgcaagtggaacaaaacaggacatgcagtcagagtcattggccgcctttcctcactggaaaacgtctacataaaggcggataagcagaagaacggtatcaaagccaatttccatatccgccacaacatcgaagatggcggagtgcagctcgcataccactaccaacagaatacgccaatcggagacggccccgtgctgctgcccgacaaccattacctgagtgttcagtcgaagcttagcaaggacccaaacgaaaaacgagatcatatggttctgctggaatttgtgactgctgctggtattaccctgggcatggatgaactctataagggtggaacgggaggctccatggttagcaaaggcgaggagctgttcactggcgtcgtgccgatcctggtcgagttggacggagatgttaatggacacaaattcagtgtgagtggcgagggcgagggcgatgcgacatacggcaagttgaccttgaagttcatttgcaccaccggtaaactgccagtaccctggcccacgctggtcaccacgctcacctatggcgtccagtgcttctctcgttatccggatcacatgaagcagcatgatttcttcaagtccgccatgcctgaggggtacattcaagagcgaacaatcttcttcaaggacgacggcaattacaagaccagagccgaagtgaagtttgaaggcgatacactagtgaaccgcatcgagctaaagggtattgattttaaggaggatggcaacatcttgggccacaagctggagtacaaccttcccgatcagctaaccgaggagcagatcgcggagttcaaggaactgttttcgctgttcgacaaagatggtgatggtacgattaccacgaaggaattgggaacagtgatgcggtccctggggcagaaccccaccgaggccgagctgcaggatatgataaacgaggtggatgctgatggtgacggcacgattgattttcccgagttcttgaccatgatggcccgaaagatgaagtaccgcgatacagaggaggagattagggaggcattcggtgtgttcgacaaggacggcaatggatacattagtgcagcagagttgcgccacgttatgactaacctgggtgagaagttgactgacgaagaggtcgatgagatgattagggaagccgatattgacggcgatggccaggtgaactatgaggaattcgtacaaatgatgaccgccaaagagggtcgcggatcgctgctgacctgcggcgacgtggaggaaaaccccggccccgttagcaagggtgaagaggtgattaaagaattcatgcgttttaaagtacgcatggagggtagcatgaacggtcatgagtttgagatcgagggcgaaggcgaaggtcgtccgtacgagggtacgcagaccgctaaactgaaagtaaccaaaggcggtcctctgccgttcgcgtgggacatcctgtccccgcagtttatgtatggttccaaggcctacgtcaaacacccagccgatatcccggactacaagaagctgtctttcccggagggtttcaagtgggagcgtgtaatgaactttgaggacggtggcctggttaccgtcacccaggatagctctctgcaagatggcaccctgatctacaaggtgaagatgcgtggtacgaactttccgccggatggcccggtgatgcagaaaaagacgatgggatgggaagcatctaccgagcgcctgtatccgcgtgacggtgttctgaaaggtgagatccaccaggcgctgaaactgaaggacggcggtcactacctggttgaatttaagacgatctatatggctaaaaagccggttcaactgccaggttactactacgtggacaccaaactggatatcacgtctcacaacgaagattataccatcgtcgaacagtatgaacgtagcgagggccgtcaccacctgttcctgggccacggtactggttctacgggttctggttcctctggcactgcgtcttccgaagacaacaacatggcggtgatcaaagagttcatgaggttcaaagtacgtatggaaggttctatgaacggccatgagttcgagattgaaggtgagggcgagggtcgcccatacgagggcactcagaccgcaaagctgaaagttaccaaaggtggcccactgccttttgcgtgggatatcctgtctccgcagttcatgtacggttccaaagcctacgtaaaacacccggcagacatcccggattacaaaaagctgtccttcccagagggcttcaaatgggaacgtgttatgaatttcgaagatggcggcctggtcactgttacgcaggacagcagcctgcaagacggtactctgatttacaaagtgaaaatgcgtggcactaacttcccgcctgacggtccagtaatgcagaagaaaactatgggctgggaagctagcaccgaacgtctgtacccgcgcgacggtgtgctgaaaggcgagattcaccaggccctgaagctgaaggatggtggtcactatctggttgagttcaaaactatctacatggcaaaaaagcctgtacaactgccgggctattactacgttgacactaaactggacatcacctcccacaacgaggattatactattgttgaacagtacgagcgttctgaaggtcgccatcatctgtttctgtatggtatggacgaactgtataaataaggtgtacaaatctagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccggccgtttaaacgaattcttgaagacgaaagggcctcgtgatacgcctatttttataggttaatgtcatgataataatggtttcttagactcaggtggcacttttcggggaaaggttgcttaagaataagtaattcttaagcaaccattacgccaagcttgcatgcaggcctctgcagtcgacgggcccgggatccgatcgacaagccgaacatatgggcgcgccgggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaatttcgaagatggtggagttgtcacagtcacacaagattcttcgctccaagatggttgttttatttacaaagtgaaatttattggagtcaattttccgtcggatggaccggttatgcaaaagaaaacaatgggttgggaagcatcgacggaacgtctgtatccacgagatggagttttgaaaggcgaaattcacaaagctcttaaactcaaagatggcggacattaccttgtagaatttaaaagcatttatatggctaagaaaccggtccaactccctggatattattatgtcgattcgaaactggatattacttcacataacgaagattatacgatagttgagcaatatgaacgcactgaaggtcgtcaccatctctttctctaaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatggcgcgcctccggaacataatggtgcatctagatgcattcgcgaggtaccgagctcgaattcactggggttgcttaagaataagtaattcttaagcaacccaaccccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 23 - DNA, 14652 bp, Synthetic construct
  source 1..14652: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggaggctcgcgtatcggtgattcattctgctaaccagtaaggcaaccccgccagcctagccgggtcctcaacgacaggagcacgatcatgcgcacccgtggccagggccgcaagcttgcatgcctgcaggtcggagtactgtcctccgagcggagtactgtcctccgagcggagtactgtcctccgagcggagtactgtcctccgagcggagtactgtcctccgagcggagactctagccctagggcatgcctgcaggtcggagtactgtcctccgagcggagtactgtcctccgagcggagtactgtcctccgagcggagtactgtcctccgagcggagtactgtcctccgagcggagactctagcgctagcgcatgcctgcaggtcggagtactgtcctccgagcggagtactgtcctccgagcggagtactgtcctccgagcggagtactgtcctccgagcggagtactgtcctccgagcggagactctagcactagtgcatgcctgcaggtcggagtactgtcctccgagcggagtactgtcctccgagcggagtactgtcctccgagcggagtactgtcctccgagcggagtactgtcctccgagcggagactctagcgacgtcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagatctaaaaggtaggttcaaccactgatgcctaggcacaccgaaacgactaaccctaattcttatcctttacttcaggcggccgcaacttaaaaaaaaaaatcaaaatgggctcacatcatcaccaccaccacggaatggctagcatgacgggcggacaacagatgggccgcgatctgtacgatgatgatgataaggacctggcgacaatggttgacagcagccggcgcaagtggaacaaaacaggacatgcagtcagagtcattggccgcctttcctcactggaaaacgtctacataaaggcggataagcagaagaacggtatcaaagccaatttccatatccgccacaacatcgaagatggcggagtgcagctcgcataccactaccaacagaatacgccaatcggagacggccccgtgctgctgcccgacaaccattacctgagtgttcagtcgaagcttagcaaggacccaaacgaaaaacgagatcatatggttctgctggaatttgtgactgctgctggtattaccctgggcatggatgaactctataagggtggaacgggaggctccatggttagcaaaggcgaggagctgttcactggcgtcgtgccgatcctggtcgagttggacggagatgttaatggacacaaattcagtgtgagtggcgagggcgagggcgatgcgacatacggcaagttgaccttgaagttcatttgcaccaccggtaaactgccagtaccctggcccacgctggtcaccacgctcacctatggcgtccagtgcttctctcgttatccggatcacatgaagcagcatgatttcttcaagtccgccatgcctgaggggtacattcaagagcgaacaatcttcttcaaggacgacggcaattacaagaccagagccgaagtgaagtttgaaggcgatacactagtgaaccgcatcgagctaaagggtattgattttaaggaggatggcaacatcttgggccacaagctggagtacaaccttcccgatcagctaaccgaggagcagatcgcggagttcaaggaactgttttcgctgttcgacaaagatggtgatggtacgattaccacgaaggaattgggaacagtgatgcggtccctggggcagaaccccaccgaggccgagctgcaggatatgataaacgaggtggatgctgatggtgacggcacgattgattttcccgagttcttgaccatgatggcccgaaagatgaagtaccgcgatacagaggaggagattagggaggcattcggtgtgttcgacaaggacggcaatggatacattagtgcagcagagttgcgccacgttatgactaacctgggtgagaagttgactgacgaagaggtcgatgagatgattagggaagccgatattgacggcgatggccaggtgaactatgaggaattcgtacaaatgatgaccgccaaagagggtcgcggatcgctgctgacctgcggcgacgtggaggaaaaccccggccccgttagcaagggtgaagaggtgattaaagaattcatgcgttttaaagtacgcatggagggtagcatgaacggtcatgagtttgagatcgagggcgaaggcgaaggtcgtccgtacgagggtacgcagaccgctaaactgaaagtaaccaaaggcggtcctctgccgttcgcgtgggacatcctgtccccgcagtttatgtatggttccaaggcctacgtcaaacacccagccgatatcccggactacaagaagctgtctttcccggagggtttcaagtgggagcgtgtaatgaactttgaggacggtggcctggttaccgtcacccaggatagctctctgcaagatggcaccctgatctacaaggtgaagatgcgtggtacgaactttccgccggatggcccggtgatgcagaaaaagacgatgggatgggaagcatctaccgagcgcctgtatccgcgtgacggtgttctgaaaggtgagatccaccaggcgctgaaactgaaggacggcggtcactacctggttgaatttaagacgatctatatggctaaaaagccggttcaactgccaggttactactacgtggacaccaaactggatatcacgtctcacaacgaagattataccatcgtcgaacagtatgaacgtagcgagggccgtcaccacctgttcctgggccacggtactggttctacgggttctggttcctctggcactgcgtcttccgaagacaacaacatggcggtgatcaaagagttcatgaggttcaaagtacgtatggaaggttctatgaacggccatgagttcgagattgaaggtgagggcgagggtcgcccatacgagggcactcagaccgcaaagctgaaagttaccaaaggtggcccactgccttttgcgtgggatatcctgtctccgcagttcatgtacggttccaaagcctacgtaaaacacccggcagacatcccggattacaaaaagctgtccttcccagagggcttcaaatgggaacgtgttatgaatttcgaagatggcggcctggtcactgttacgcaggacagcagcctgcaagacggtactctgatttacaaagtgaaaatgcgtggcactaacttcccgcctgacggtccagtaatgcagaagaaaactatgggctgggaagctagcaccgaacgtctgtacccgcgcgacggtgtgctgaaaggcgagattcaccaggccctgaagctgaaggatggtggtcactatctggttgagttcaaaactatctacatggcaaaaaagcctgtacaactgccgggctattactacgttgacactaaactggacatcacctcccacaacgaggattatactattgttgaacagtacgagcgttctgaaggtcgccatcatctgtttctgtatggtatggacgaactgtataaataaggtgtacaaatctagaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtgtactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacggccggccgtttaaacgaattcttgaagacgaaagggcctcgtgatacgcctatttttataggttaatgtcatgataataatggtttcttagactcaggtggcacttttcggggaaaggttgcttaagaataagtaattcttaagcaaccattacgccaagcttgcatgcaggcctctgcagtcgacgggcccgggatccgatcgacaagccgaacatatgggccagtgaattcgagctcggtacctcgcgaatgcatctagatgcaccattatgttccggaggcgcgccatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctttagagaaagagatggtgacgaccttcagtgcgttcatattgctcaactatcgtataatcttcgttatgtgaagtaatatccagtttcgaatcgacataataatatccagggagttggaccggtttcttagccatataaatgcttttaaattctacaaggtaatgtccgccatctttgagtttaagagctttgtgaatttcgcctttcaaaactccatctcgtggatacagacgttccgtcgatgcttcccaacccattgttttcttttgcataaccggtccatccgacggaaaattgactccaataaatttcactttgtaaataaaacaaccatcttggagcgaagaatcttgtgtgactgtgacaactccaccatcttcgaaattcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggggtggcgaccggtggatcgtaccgtcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttaatggtcacttggttgttcacgatcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctagcgcacacggacaatggacccgacaaattctacgccaaggatttaatgataatgtcgggcaacgtatccgttcattttatcaataacctacaaaaatgtcgcgcgcatcacaaagacatcgacgcgcgtagaattctacccgtaaagcgagtttagttatgagccatgtgcaaaacatgacatcatctcgatttgaaaaacaaatgacatcatccactgatcgtgcgttacaagtagaattctactcgtaaagccagttcggttatgagccgtgtgcaaaacatgacatcagcttatgactcatacttgattgtgttttacgcgggcgcgtaagagctcggcctccggaacataatggtgcatctagatgcattcgcgaggtaccgagctcgaattcactggggttgcttaagaataagtaattcttaagcaacccaaccccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 24 - DNA, 7985 bp, Synthetic construct
  source 1..7985: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggaccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctggagcgagaccggagtcgtcttcgctttccgaattcgagctcggtacctcgcgaatgcatctagatatcagatctggatcccgggcccgtcgactgcagaggcctgcatgcaagcttgataacttgtgaagacgaccatggtctcaccatcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 25 - DNA, 12462 bp, Synthetic construct
  source 1..12462: mol_type = other DNA
gactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaaatcgtcacctgcaggaaggtttaaacgcatttaggtgacactatagaagtgtgtatcgctcgagggatccgaattcgaagtcttggtacggaggggatccgatcgacaagccgaacatatgggttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcggggcctcaactttgtataataaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaatggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattatcgatacaccgggccgtcgcatggttattccacttgcgtctgcgcgcttgctctcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataatgtcaggctcccttatacacagccagtctgcacgtcgcatagtgactggatatgttgtgttttacagtattatgtagtctgttttttatgcaaaatctaatttaatatattgatatttatatcattttacgtttctcgttcaacttttctatacaaagttgtccagctcttcctactgcaccgagctcttacgcgcccgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgcgtcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtttccagactttacgaaacacggaaaccgaagaccattcatgttgttgctcaggtcgcgctcgcgtatcggtgattcattctgctaaccagtaaggcaaccccgccagcctagccgggtcctcaacgacaggagcacgatcatgcgcacccgtggccagcggccgcgtttaaacaccggttagggatgccaactcgtgcttttaagcgcgtaaagttttcgattcggcgccaactctccatcgcatttaaataaataatttctcgaggacagatctaaacgagtttttaagcaaactcactccctgacaataaaaacgcaattatctttaaataaatatattctatttattaaccaagtagctaatatagctaaaatcaatttgttgctaacttaaaactaacataattatgtattagttatttacattcttttggttggttacaaccaaataaataatatcacttttaaaaacttaagccaggaactgaataattttctatttggctttagtcgaccctagggtttaaacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatatatcaaattgtggcattttgattgctagcggtacctgagttcttctttctcggtaacttgttgaaagtattcagagttctcttcttgtattcaataattacttcttggcagatttcagtagttgcagttgatttacttggttgctggttacttttaattgattcactttaacttgcactttactgcagattgtttagcttgttcagctgcgcttgtttatttgcttagctttcgcttagcgacgtgttcactttgcttgtttgaattgaattgtcgctccgtagacgaagcgcctctatttatactccggcgctcttttcgcgaacattcgaggcgcgctctctcgaaccaacgagagcagtatgtcgtttactgtgtgacagagtgagagagcattagtgcagagagggagagacccaaaaagaaaagagacaataacgaataacggccagagaaatttctcgagttttctttctgccaaacaaatgacctaccacaataaccagtttgttttgggattcagatctggccggccgaattcttgaagacgaaagggcctcgcaactttgtatacaaaagttgaacgagaaacgtaaaatgatataaatatcaatatattaaattagattttgcataaaaaacagactacataatactgtaaaacacaacatatccagtcactatgggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcgcatagtgactggatatgttgtgttttacagtattatgtagtctgttttttatgcaaaatctaatttaatatattgatatttatatcattttacgtttctcgttcaactttttaatacaaagttgcctccggaacataatggtgcatctagatgcccattcctgtagacttcttaattaagacgtcagaattctcgaggcggccgcatgtgagtctccctatagtgagtcgtattaatttcgcgggcgcgtcagcgggagagccgttgtaaggcggca

SEQ ID NO: 26 - DNA, 59 bp, Synthetic construct
  source 1..59: mol_type = other DNA
gtctcgtgggctcggagatgtgtataagagacagagctggacaactttgtatagaaaag

SEQ ID NO: 27 - DNA, 59 bp, Synthetic construct
  source 1..59: mol_type = other DNA
gtctcgtgggctcggagatgtgtataagagacagcgtctcacaactttgtatacaaaag

SEQ ID NO: 28 - DNA, 62 bp, Synthetic construct
  source 1..62: mol_type = other DNA
gtctcgtgggctcggagatgtgtataagagacagcgagtataatcaactttgtatacaaaag

SEQ ID NO: 29 - DNA, 60 bp, Synthetic construct
  source 1..60: mol_type = other DNA
gtctcgtgggctcggagatgtgtataagagacagaagcaacccaactttgtatacaaaag

SEQ ID NO: 30 - DNA, 62 bp, Synthetic construct
  source 1..62: mol_type = other DNA
gtctcgtgggctcggagatgtgtataagagacagaacaactagtcaactttgtatacaaaag

SEQ ID NO: 31 - DNA, 61 bp, Synthetic construct
  source 1..61: mol_type = other DNA
gtctcgtgggctcggagatgtgtataagagacagctctctagtcaactttgtatacaaaag

SEQ ID NO: 32 - DNA, 60 bp, Synthetic construct
  source 1..60: mol_type = other DNA
gtctcgtgggctcggagatgtgtataagagacagctcaccatcaactttgtatacaaaag

SEQ ID NO: 33 - DNA, 57 bp, Synthetic construct
  source 1..57: mol_type = other DNA
gtctcgtgggctcggagatgtgtataagagacagctcggcaactttgtatacaaaag

SEQ ID NO: 34 - DNA, 415 bp, Synthetic construct
  source 1..415: mol_type = other DNA
aaataatgattttattttgactgatagtgacctgttcgttgcaacaaattgataagcaatgcttttttataatgccaactttgtataataaagttgctttgagtgtgtgtagacatcaaggggacgtagctcagtggtagagcgctcgcttcgcatgtgagaagtcccgggttcaaaccccggcgtctccaaccctcatcccggtcctaatgttttagagctagaaatagcaagttaaaataaggctagtccgttatcaacttgaaaaagtggcaccgagtcggtgctaacaaagcaccagtggtctagtggtagaatagtaccctgccacggtacagacccgggttcgattcccggctggtgcattttttgcctacctggagcctgagagttgttcaataaaataaaaatgttt

SEQ ID NO: 35 - DNA, 97 bp, Synthetic construct
  source 1..97: mol_type = other DNA
caacttttctatacaaagttggcattataagaaagcattgcttatcaatttgttgcaacgaacaggtcactatcagtcaaaataaaatcattatttg

SEQ ID NO: 36 - DNA, 97 bp, Synthetic construct
  source 1..97: mol_type = other DNA
caaataatgattttattttgactgatagtgacctgttcgttgcaacaaattgatgagcaatgcttttttataatgccaactttgtatacaaaagttg

SEQ ID NO: 37 - DNA, 97 bp, Synthetic construct
  source 1..97: mol_type = other DNA
caactttttaatacaaagttggcattataagaaagcattgcttatcaatttgttgcaacgaacaggtcactatcagtcaaaataaaatcattatttg

SEQ ID NO: 38 - DNA, 1013 bp, Synthetic construct
  source 1..1013: mol_type = other DNA
aaataatgattttattttgactgatagtgacctgttcgttgcaacaaattgataagcaatgcttttttataatgccaactttgtataataaagttgctttgagtgtgtgtagacatcaaggggacgtagctcagtggtagagcgctcgcttcgcatgtgagaagtcccgggttcaaaccccggcgtctccatttcaaatgctgcctaacacgttttagagctagaaatagcaagttaaaataaggctagtccgttatcaacttgaaaaagtggcaccgagtcggtgctaacaaagcaccagtggtctagtggtagaatagtaccctgccacggtacagacccgggttcgattcccggctggtgcattttttgcctacctggagcctgagagttgttcaataaaataaaaatgtttggtatacatatatctttcttctttcgtggggagttttgttgcttttgttttgtacactatggggttaagactttcctttgaataacagtttataatgaactggtatagccttaaacttaaaaattataagaagggcctgtatttatatggtctttatatctcgtatttgatcatttcttatgtttaagtttgggtaaaatttaaaaaaaaaaattttaaaaagcaaactaagaatgtcaaaatttagatgcttgaatgtttataacacttgtaactctttgcaaggcattacatctgtatagcaaatattaatcatttattgtatactgtactaaaataaatgaaattaaactccattcacttataagtaaactgctgtcactgcatcttcatttctgctaggttttttgcaggtcaaagctctgaagcgtcaaccccctggtggctcccccccccccatacaatcaatcaaccggcaaaaaatttcaaatgctgcctaaccaacttttctatacaaagttggcattataagaaagcattgcttatcaatttgttgcaacgaacaggtcactatcagtcaaaataaaatcattatttg

SEQ ID NO: 39 - DNA, 695 bp, Synthetic construct
  source 1..695: mol_type = other DNA
caaataatgattttattttgactgatagtgacctgttcgttgcaacaaattgatgagcaatgcttttttataatgccaactttgtatacaaaagttgactggcactagaacaaaagctttggcgaaagctgcgctggcagctcgtccatatgatcgtcactctcaatttagtttataaacaacgctcgccggctgcagttggaaacggagagcgcgtaatcggcgatatcaattcgtaggacataggaccgccgtcagcatatgtaattgcttatcacagtctgactgtccgaaatcgctccagcgacaacagatcccccaacaactcgtgccaccccccgcaaattgccggcttacgtaagccaattgtattcgttttagctgagagaacatcaatcaaaatacaaataacaaaaaaacatgcggctattggccagacacgcgattcgattgctgggtcaggagaatagcgcgggcgaggtggcttcactttcgagaggagcaatccggctgaaggccacaaccggttacctcaatctggccacggcaagtgttcagccattggagccggagaaacaggtgctgagaaaggtgagtacaactttttaatacaaagttggcattataagaaagcattgcttatcaatttgttgcaacgaacaggtcactatcagtcaaaataaaatcattatttg

SEQ ID NO: 40 - DNA, 2680 bp, Synthetic construct
  source 1..2680: mol_type = other DNA
ggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtgtcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaatgtcaggctcccttatacacagccagtctgcacgtcg

(19) United States
(12) Patent Application Publication
(10) Pub. No.: US 2024/0409947 A1
(43) Pub. Date: Dec. 12, 2024

(54) VECTORS AND METHODS FOR EFFICIENT CLONING AND HOMOLOGY DIRECTED REPAIR

(71) Applicant: HOWARD HUGHES MEDICAL INSTITUTE, Chevy Chase, MD (US)

(72) Inventor: David L. Stern, Leesburg, VA (US)

(21) Appl. No.: 18/736,729

(22) Filed: Jun. 7, 2024

Related U.S. Application Data
(60) Provisional application No. 63/507,335, filed on Jun. 9, 2023.

(51) Int. Cl. C12N 15/65; C12N 9/22; C12N 15/11

(52) U.S. Cl. CPC C12N 15/65; C12N 9/22; C12N 15/111; C12N 2310/20

(57) ABSTRACT

Vectors and methods are described for efficient cloning and integration of a DNA sequence of interest into a genome of a cell. Vectors include a cassette having a nucleotide sequence having a negative selection marker that is a ccdB gene, which is flanked by non-identical attR recombination recognition sequences.

### RELATED APPLICATIONS

This application claims priority from U.S. Provisional Application Ser. No. 63/507,335 filed Jun. 9, 2023, the entire disclosure of which is incorporated herein by this reference.

### REFERENCE TO AN ELECTRONIC SEQUENCE LISTING

The contents of the electronic sequence listing (Stern-18074N-23019-sequence-listing.xml); Size: 321,778 bytes; and Date of Creation: Jun. 6, 2024) is herein incorporated by reference in its entirety.

### TECHNICAL FIELD

The presently-disclosed subject matter generally relates to vectors and methods for efficient cloning and integration of a DNA sequence of interest into a genome of a cell.

### INTRODUCTION

Homology-directed repair (HDR) is a powerful tool for modifying genomes in precise ways to address many biological questions. Clustered Regularly Interspersed Short Palindromic Repeats (CRISPR)-Cas induced mutagenesis has revolutionized experimental approaches in model and non-model organisms. DNA cuts induced by Cas9 or other enzymes^{1,2} can be repaired by the non-homologous end-joining pathway to induce small deletions or, if a homologous DNA template is present, then cuts may be repaired by HDR³.

HDR is normally implemented by introduction of three separate elements: Cas-9 (or analogous enzyme), short guide RNA (sgRNA), and homologous DNA. Usually, these three reagents are introduced as distinct components. In the most efficient systems, a source of sgRNA and the homology arms are typically cloned into separate plasmids, often with a “payload,” or DNA that is intended to be inserted into a genome by HDR, located between flanking homology arms. Usually, plasmids containing homology arms are constructed by PCR of homology arms followed by cloning of arms together with the payload.

While HDR provides a great power to manipulate gene function and to introduce new reagents into the germ line of experimental organisms, existing platforms for CRISPR-Cas-mediated HDR in many species involve multiple cloning steps and have low HDR efficiency.

Accordingly, there remains a need in the art for vectors and methods that allow for simplified cloning and increased HDR efficiency.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes vectors (also referred to herein as plasmids) and methods that allow for simplified cloning and increased efficiency. In some embodiments, the vectors and methods can be used for CRISPR-Cas-mediated HDR in many species, with simplified cloning steps and increased HDR efficiency.

To simplify cloning of HDR plasmids, in some embodiments of the presently-disclosed subject matter, a unique plasmid platform has been designed and is disclosed herein, and is referred to herein as the Janelia Atalanta (pJAT) series. The pJAT series beneficially and inventively exploits recent advances in dsDNA synthesis.

In some embodiments, the availability of cost-effective long dsDNA fragments is employed to facilitate Gateway® cloning of gRNA sequences and homology arms in one step. In particular, embodiments of the pJAT series were designed to include two Gateway® cloning sites for simultaneous introduction of synthesized fragments.

The Gateway® cloning system is a site-specific recombination technology that takes advantage of the att site-specific recombination properties of bacteriophage lambda to provide a rapid and efficient way to move a gene of interest between multiple vector systems. Additional information can be found in the following references, which are incorporated herein by this reference: U.S. Pat. Nos. 5,888,732, 6,143,557, 6,171,861, 6,270,969, 6,277,608, and 6,720,140; and the Gateway® Technology manual, updated Sep. 22, 2003; available at assets.thermofisher.com/TFS-Assets/LSG/manuals/gatewayman.pdf).

It was discovered when attempting to build a dual-Gateway® plasmid following earlier examples that multiple copies of the standard Gateway® negative selection marker, ccdB, were unstable⁴. As disclosed herein, this challenge was overcome by the clever introduction of a unique dual selection system to ensure efficient simultaneous cloning of two attL cassettes.

In some embodiments, flanking the Gateway®-like cloning sites, on one or both ends, a promoter sequence was used to drive RNA transcription. The synthesized RNA downstream of the promoter contains tRNA sequences flanking the sgRNA sequence, followed by a U6 terminator, providing efficient precise release of sgRNA molecules⁵. Any payload can then be placed between the Gateway®-like cloning sites, and a series of plasmids carrying some commonly used payloads have been generated as examples.

While the plasmids of the presently-disclosed subject matter were designed for efficient cloning, it was unexpectedly and surprisingly discovered that the plasmids yielded far higher HDR integration efficiencies on average than has been observed by us and others using other methods^{3,6,7}. Therefore, these plasmids have been named the Janelia Atalanta (pJAT) series, after the mythological Greek heroine and exceptional archer. The high efficiency of HDR with pJAT plasmids inspired attempts of new kinds of experiments that have not previously been feasible in certain species, such as introduction of chromosomal inversions.

The pJAT plasmids are modular and generic. One or two promoters from any species can be introduced outside the Gateway®-like cloning cassettes and any payload can be introduced between the cloning cassettes. pJAT plasmids can therefore be of value for HDR experiments in many model and non-model organisms.

The presently disclosed subject matter includes a cloning vector, which includes a first cassette having a nucleotide sequence comprising a first negative selection marker that is a ccdB gene, which is flanked by first non-identical attR recombination recognition sequences. In some embodiments, the cloning vector further includes a second cassette having a nucleotide sequence comprising a second negative selection marker that is not a ccdB gene, which is flanked by second non-identical attR recombination recognition sequences, wherein the first non-identical attR recombination recognition sequences are distinct from the second non-identical attR recombination recognition sequences.

The presently-disclosed subject matter further includes a donor vector, which includes a first homology arm sequence flanked by first non-identical attR recombination recognition sequences, and a short guide RNA (sgRNA) sequence adjacent the first or the second homology arm sequence and separated from the homology arm by an RNA polymerase termination sequence. Some embodiments of the donor vector further include a second homology arm sequence flanked by second non-identical attR recombination recognition sequences, wherein the first non-identical attR recombination recognition sequences are distinct from the second non-identical attR recombination recognition sequences.

The presently-disclosed subject matter also includes a method of preparing a donor vector by simultaneously introducing two DNA fragments of interest into a cloning vector, which includes the steps of (a) providing the cloning vector as disclosed herein; (b) incubating the cloning vector with a first DNA fragment of interest flanked by attL recombination sites, a second DNA fragment of interest flanked by attL recombination sites, and a recombination enzyme; and (c) selecting a vector in which the first DNA fragment of interest has replaced the first cassette, and the second DNA fragment of interest has replaced the second cassette, thereby obtaining the donor vector.

The presently-disclosed subject matter also includes a method of integrating a donor DNA into a genome of a cell, which includes the steps of introducing a donor vector as disclosed herein into a cell; and providing to the cell a source of an enzyme for creating double-strand breaks (DSBs) guided by the sgRNA encoded in the donor vector. In some embodiments, the method of integrating a donor DNA into a genome of a cell also involves the step of confirming an efficiency of integration of greater than about 5, 10, 15, 20, 25, 30, 35, 40, 45, or 50%.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1D. Design and use of exemplary Janelia Atalanta plasmids. FIG. 1A: General design of the Janelia Atalanta series. Each plasmid contains two Gateway®-compatible cloning sites, flanked on the left and right, respectively, by attR3-attR4 and attR5-attR6 sites. Each Gateway®-compatible cassette contains a different positive and negative selection marker. The negative selection markers ccdB and SacB can be selected simultaneously by cloning into cells of any standard bacterial strain (such as DH5alpha) and plating on Tryptone-Yeast media containing a high concentration of sucrose. Each Atalanta plasmid contains one or two U6 promoters flanking the Gateway® cassettes and a payload between the Gateway® cassettes. FIG. 1B: DNA fragments or plasmids containing paired attL3-attL4 and attL5-attL6 sites can be cloned into the base Atalanta plasmid simultaneously. In this example, synthetic DNA fragments containing left and right homology arms and a tRNA-sgRNA-tRNA array are cloned into the Gateway® sites. The plasmid thus provides a source both of sgRNA and of homology arms for homology directed repair. FIG. 1C: An example of an Atalanta plasmid after cloning of Gateway®-compatible arms that is ready for injection into Drosophila embryos. Cas9 is provided either through co-injection of Cas9 mRNA or protein or by injection into embryos expressing Cas9. FIG. 1D: Detail of the components of a generic tRNA-sgRNA-tRNA region cloned downstream of the U6 promoter.

FIG. 2A-2D. Payloads of exemplary Janelia Atalanta plasmids. FIG. 2A: A set of plasmids containing fluorescent protein markers, attP sites, and removable fluorescent protein markers useful for targeted mutagenesis and generation of attP landing sites. FIG. 2B: Plasmids to facilitate cloning of novel payloads or different RNA transcription promoters. MCS=Multiple Cloning Site. FIG. 2C: Plasmids carrying various effectors, including nanos-Cas9 (pJAT17 (SEQ ID NO: 1)), B3 integrase under heat shock control (pJAT43 (SEQ ID NO: 2)), and two plasmids expressing vasa-phiC31 integrase with different fluorescent protein markers (pJAT56 (SEQ ID NO: 3), pJAT62 (SEQ ID NO: 4)). FIG. 2D: Plasmids carrying split-GFP constructs under three alternative promoter systems for construction of chromosomal inversions.

FIGS. 3A and 3B—Integration efficiencies of Atalanta homology directed repair payloads into Drosophila species. The percent of positive events is plotted against the number of viable crosses screened. FIG. 3A: On average, 25% of injected animals producing fertile adults yielded integration events into genes categorized as “viable” on Flybase or into non-genic regions. No obvious difference was observed in integration efficiencies between the three Drosophila species D. melanogaster, D. simulans, and D. yakuba. FIG. 3B: Integration efficiency for payloads targeting genes categorized as “lethal” on Flybase.

FIG. 4A-4D—Schematic representation of scarless genome modification using pJAT plasmid. FIG. 4A, 4B: Diagram of a pJAT plasmid illustrating the location of the synthesized pBac transposon 5′ and 3′ arms internal to the synthesized homology arms (FIG. 4A). The sgRNA, included within the tRNA-sgRNA-tRNA array on the left homology arm, targets a genomic site that includes a TTAA motif (FIG. 4B), the native target site for the pBac transposon. The synthesized right homology arm includes an additional TTAA sequence between the 3′ pBac sequence and the right homology arm. Intended modifications to the genomic sequence can be included in either or both homology arms. FIG. 4C: Representation of the genomic sequence after integration of the pJAT plasmid. Flies containing the intended genomic modification(s) can be identified by fluorescent reporter proteins encoded as a payload or by PCR and sequencing. FIG. 4D: After exposure to a source of pBac transposase, flies can be recovered that have lost the reporter gene and contain the intended scarless genome modification.

FIG. 5A-5C—Design of pJAT plasmids for targeted inversions. FIG. 5A: Schematic illustrating the position and orientation of DNA sequences flanking two cut sites before (top) and after (bottom) a chromosomal inversion. FIG. 5B: Illustration of two pJAT plasmids, each containing one half of a split-GFP reporter and a gRNA targeting one of the two intended cut sites. Each plasmid includes synthesized arms oriented in the direction of the intended inversion so that homology directed repair at both sites simultaneously will tend to drive an inversion. FIG. 5C: Illustration of three inversions targeted on the right arm of chromosome 3 in D. yakuba. The positions of the fruitless (fru) and Doublesex (dsx) genes and the locations of target gRNA sites in the genes are depicted. The three targeted inversions are also shown.

FIG. 6—Effect of tRNA-sgRNA-tRNA array on CRISPR-Cas9 HDR efficiency. Three plasmids were created where the left homology arm contained either no tRNA and sgRNA (Construct Form A), just the sgRNA (Construct Form B), or the full tRNA-sgRNA-tRNA array (Construct Form C). Injections of the plasmid without a sgRNA were supplemented with in vitro transcribed sgRNA. All plasmids targeted the CG42402 gene. The table shows the HDR efficiency for each construct type.

FIG. 7—To test the hypothesis that in vivo linearization of the template DNA increases the efficiency of HDR, plasmids targeting two loci were constructed either without (Construct Form A) or with (Construct Form A) CRISPR target sites for the sgRNA encoded in the tRNA-sgRNA-tRNA array. Results are shown in the table, which illustrates that plasmids containing the flanking CRISPR sites did not increase HDR efficiency for two genes.

FIG. 8—To test the hypothesis that sgRNA concentration limited HDR, plasmids including either one (Construct Form A) or two (Construct Form B) sources of sgRNA were constructed. Results are shown in the table, which illustrates that plasmids containing two sources of sgRNA did not increase HDR efficiency for two genes on average.

FIGS. 9A and 9B—Sequence confirmation of scarless genome mutagenesis. FIG. 9A: Sequencing of genomic DNA containing inserted pBac transposable element with internal MHC-DsRed reporter. Left homology arm includes intended 2 single bp changes. PCR fragments were generated with primers external to homology arms and internal to the pBac transposon and Sanger sequenced with internal primers. Sanger sequencing products were aligned to the original genome sequence and mismatches are shown in the Identity track and below the Sanger sequencing read. FIG. 9B: A PCR product of the final scarless allele generated with primers shown was Nanopore sequenced and aligned to the original genome sequence and mismatches are shown in the Identity track and within the consensus Nanopore sequencing read.

FIG. 10A-10F—Detailed maps from the D. yakuba genome of the gRNA sites, homology arms, intended inversion ends, and PCR primers for the genes VMS100-2 (FIG. 10A), fray (FIG. 10B), Os-C (FIG. 10C), and Ids (FIG. 10D).

FIG. 11A-11F—Sequences of PCR products generated using primers illustrated in FIG. 8 on strains carrying the putative inversions. Three inversions were generated, one between VMS100-2 and fray (FIG. 11A, 11B), one between Osc and Ids (FIG. 11C, FIG. 11D), and one between VMS100-2 and Ids (FIG. 11E, FIG. 11F).

### BRIEF DESCRIPTION OF THE SEQUENCE LISTING

SEQ ID NO: 1 is an example of a sequence for a vector of the Janelia Atalanta (pJAT) series of vectors as disclosed herein, which is referred to as pJAT17{dU6-3-attR3-Amp-ccdB-attR4-nos-Cas9-nos3′UTR-3×P3-EYFP-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6-attR recombination recognition sequences; Amp—ampicillin resistance selection marker; ccdB—control of cell death B gene used as a negative selection marker; Cas9—gene encoding the Cas9 endonuclease enzyme; nos3′UTR—3′ untranslated region from the Drosophila melanogaster nanos gene used as a terminator; nos—nanos gene promoter; 3×P3—three copies of the Pax-6 binding sites, serving as a promoter; EYFP—enhanced yellow fluorescent protein; Chlor—chloramphenicol resistance selection marker; SacB—sacB gene used as a negative selection marker. The components of the payload or donor DNA of the sequence are underlined.

SEQ ID NO: 2 is another example of a sequence for a vector of the pJAT series, which is referred to as pJAT43{dU6-3-attR3-Amp-attR4-ccdB-ie1-EGFP-HES-B3-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; ie1—ie1 promotor; EGFP—enhanced green florescent protein; HES—HES promoter; B3—B3 integrase under heat shock control; Chlor—chloramphenicol resistance selection marker; SacB—sacB gene used as a negative selection marker; The components of the payload or donor DNA of the sequence are underlined.

SEQ ID NO: 3 is another example of a sequence for a vector of the pJAT series, which is referred to as pJAT56{dU6-3-attR3-Amp-ccdB-attR4-vas-phiC31-vasUTR, 3×P3-EGFP-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; vas—vasa promoter; phiC31—phiC31 integrase; vas UTR-3′ untranslated region of the vasa gene used to promote efficient translation; 3×P3—three copies of the Pax-6 binding sites, serving as a promoter; EGFP—enhanced green florescent protein; Chlor—chloramphenicol resistance selection marker; SacB—sacB gene used as a negative selection marker. The components of the payload or donor DNA of the sequence are underlined.

SEQ ID NO: 4 is another example of a sequence for a vector of the pJAT series, which is referred to as pJAT62{dU6-3-attR3-Amp-ccdB-attR4-vas-phiC31-vasUTR, ie1-EGFP-attR5-Chlor-SacB-attR6}.The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; vas—vasa promoter; phiC31—phiC31 integrase; vas UTR-3′ untranslated region of the vasa gene used to promote efficient translation; ie1—ie1 promotor; EGFP—enhanced green florescent protein. The components of the payload or donor DNA of the sequence are underlined.

SEQ ID NO: 5 is another example of a sequence for a vector of the pJAT series, which is referred to as pJAT77{attR3-Amp-ccdB-attR4-MCS-attR5-Chlor-SacB-attR6}. The abbreviations used to describe components of the sequence are commonly used in the art: attR3, attR4, attR5, and attR6—attR recombination recognition sequences; Amp—ampicillin resistance selection marker; ccdB—control of cell death B gene used as a negative selection marker; MCS—multiple cloning site; Chlor—chloramphenicol resistance selection marker; SacB—sacB gene used as a negative selection marker. The components of the payload or donor DNA of the sequence are underlined.

SEQ ID NO: 6 is another example of a sequence for a vector of the pJAT series, which is referred to as pJAT10{dU6-3-attR3-Amp-ccdB-attR4-Mhc-dsRed-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; Mhc—myosin heavy chain promoter; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 7 is another example of a sequence for a vector of the pJAT series, which is referred to as pJAT21{dU6-3-attR3-Amp-ccdB-attR4-ie1-GFPn-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; ie1—ie1 promotor; GFPn—N-terminal fragment of green fluorescent protein; Chlor—chloramphenicol resistance selection marker; SacB—sacB gene used as a negative selection marker; The components of the payload or donor DNA of the sequence are underlined.

SEQ ID NO: 8 is another example of a sequence for a vector of the pJAT series, which is referred to as pJAT25{dU6-3-attR3-Amp-ccdB-attR4-ie1-MBP-GFPc-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; ie1—ie1 promotor; MBP—maltose-binding protein; GFPc—C-terminal fragment of green fluorescent protein; Chlor—chloramphenicol resistance selection marker; SacB—sacB gene used as a negative selection marker. The components of the payload or donor DNA of the sequence are underlined.

SEQ ID NO: 9 is another example of a sequence for a vector of the pJAT series, which is referred to as pJAT7{dU6-3-attR3-Amp-ccdB-attR4-attP-B3-3×P3-dsRed-B3-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; attP—attP recombination recognition sequence; B3—B3 integrase under heat shock control; 3×P3—three copies of the Pax-6 binding sites, serving as a promoter; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 10 is another example of a sequence for a vector of the pJAT series, which is referred to as pJAT11{dU6-3-attR3-Amp-ccdB-attR4-attP-B3-ie1 dsRed-B3-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; attP—attP recombination recognition sequence; B3—B3 integrase under heat shock control; ie1—ie1 promotor; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 11 is another example of a sequence for a vector of the pJAT series, which is referred to as pJAT18{dU6-3-attR3-Amp-ccdB-attR4-MHC-GFPn-SV40-attR5-Chlor-SacB-attR6}.The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; Mhc—myosin heavy chain promoter; GFPn—N-terminal fragment of green fluorescent protein; SV40—SV40 promoter; Chlor—chloramphenicol resistance selection marker; SacB—sacB gene used as a negative selection marker. The components of the payload or donor DNA of the sequence are underlined.

SEQ ID NO: 12 is another example of a sequence for a vector of the pJAT series, which is referred to as pJAT22{dU6-3-attR3-Amp-ccdB-attR4-3×P3-GFPn-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; 3×P3—three copies of the Pax-6 binding sites, serving as a promoter; GFPn—N-terminal fragment of green fluorescent protein; Chlor—chloramphenicol resistance selection marker; SacB—sacB gene used as a negative selection marker. The components of the payload or donor DNA of the sequence are underlined.

SEQ ID NO: 13 is another example of a sequence for a vector of the pJAT series, which is referred to as pJAT23{dU6-3-attR3-Amp-ccdB-attR4-3×P3-MBP-GFPc-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; 3×P3—three copies of the Pax-6 binding sites, serving as a promoter; MBP—maltose-binding protein; GFPc—C-terminal fragment of green fluorescent protein; Chlor—chloramphenicol resistance selection marker; SacB—sacB gene used as a negative selection marker. The components of the payload or donor DNA of the sequence are underlined.

SEQ ID NO: 14 is another example of a sequence for a vector of the pJAT series, which is referred to as pJAT27{dU6-3-attR3-Amp-ccdB-attR4-MHC-GFPc-attR5-Chlor-SacB-attR6}. The abbreviations used to describe components of the sequence are commonly used in the art:

dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; Mhc—myosin heavy chain promoter; GFPc—C-terminal fragment of green fluorescent protein; Chlor—chloramphenicol resistance selection marker; SacB—sacB gene used as a negative selection marker. The components of the payload or donor DNA of the sequence are underlined.

SEQ ID NO: 15 is another example of a sequence for a vector of the pJAT series, which is referred to as pJAT30{dU6-3-attR3-Amp-ccdB-attR4-Mhc-dsRed-attR5-Chlor-SacB-attR6-U6-1}. The abbreviations used to describe components of the sequence are commonly used in the art: U6-1 and dU6-3—U6 promoters derived from the first and third U6 genes in Drosophila melanogaster; attR3, attR4, attR5, and attR6—attR recombination recognition sequences; Amp—ampicillin resistance selection marker; ccdB—control of cell death B gene used as a negative selection marker; Mhc—myosin heavy chain promoter; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 16 is another example of a sequence for a vector of the pJAT series, which is referred to as pJART31{dU6-3-attR3-Amp-ccdB-attR4-attP-B3-ie1 dsRed-B3-attR5-Chlor-SacB-attR6-U6-1}. The abbreviations used to describe components of the sequence are commonly used in the art: U6-1 and dU6-3—U6 promoters derived from the first and third U6 genes in Drosophila melanogaster; attR3, attR4, attR5, and attR6—attR recombination recognition sequences; Amp—ampicillin resistance selection marker; ccdB—control of cell death B gene used as a negative selection marker; attP—attP recombination recognition sequence; B3—B3 integrase under heat shock control; ie1—ie1 promotor; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 17 is another example of a sequence for a vector of the pJAT series, which is referred to as pJAT32{dU6-3-attR3-Amp-ccdB-attR4-MCS-attR5-Chlor-SacB-attR6-U6-1}. The abbreviations used to describe components of the sequence are commonly used in the art: U6-1 and dU6-3—U6 promoters derived from the first and third U6 genes in Drosophila melanogaster; attR3, attR4, attR5, and attR6—attR recombination recognition sequences; Amp—ampicillin resistance selection marker; ccdB—control of cell death B gene used as a negative selection marker; MCS—multiple cloning site; Chlor—chloramphenicol resistance selection marker; SacB—sacB gene used as a negative selection marker. The components of the payload or donor DNA of the sequence are underlined.

SEQ ID NO: 18 is another example of a sequence for a vector of the pJAT series, which is referred to as pJAT36{attR3-Amp-ccdB-attR4-MCS-attR5-Chlor-SacB-attR6. The abbreviations used to describe components of the sequence are commonly used in the art: attR3, attR4, attR5, and attR6—attR recombination recognition sequences; Amp—ampicillin resistance selection marker; ccdB—control of cell death B gene used as a negative selection marker; MCS—multiple cloning site; Chlor—chloramphenicol resistance selection marker; SacB—sacB gene used as a negative selection marker. The components of the payload or donor DNA of the sequence are underlined.

SEQ ID NO: 19 is another example of a sequence for a vector of the pJAT series, which is referred to as pJAT39{U6-3-attR3-Amp-ccdB-attR4-MHC-GFP-attR5-Chlor-SacB-attR6-U6-1}. The abbreviations used to describe components of the sequence are commonly used in the art: U6-1 and dU6-3—U6 promoters derived from the first and third U6 genes in Drosophila melanogaster; attR3, attR4, attR5, and attR6—attR recombination recognition sequences; Amp—ampicillin resistance selection marker; ccdB—control of cell death B gene used as a negative selection marker; Mhc—myosin heavy chain promoter; GFP—green fluorescent protein; Chlor—chloramphenicol resistance selection marker; SacB—sacB gene used as a negative selection marker. The components of the payload or donor DNA of the sequence are underlined.

SEQ ID NO: 20 is another example of a sequence for a vector of the pJAT series, which is referred to as pJAT55{dU6-3-ttR3-Amp-ccdB-attR4-3×P3-ECFP,alpha-tub-pBac-Trsp-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; 3×P3—three copies of the Pax-6 binding sites, serving as a promoter; ECFP—enhanced cyan fluorescent protein; Alpha-tub—Alpha-tubulin promoter; p-Bac—PiggyBac® vector; Trsp—selenocysteine tRNA gene; Chlor—chloramphenicol resistance selection marker; SacB—sacB gene used as a negative selection marker. The components of the payload or donor DNA of the sequence are underlined.

SEQ ID NO: 21 is another example of a sequence for a vector of the pJAT series, which is referred to as pJAT57{dU6-3-attR3-Amp-ccdB-attR4-UAS-hCas9-Mhc-dsRed-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; UAS—Upstream Activating Sequence; hCas9—gene encoding human codon-optimized version of the Cas9; Mhc—myosin heavy chain promoter; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 22 is another example of a sequence for a vector of the pJAT series, which is referred to as pJAT58{dU6-3-attR3-Amp-ccdB-attR4-20×UAS-IVS-Syn21-op1-jGCaMP7s-T2A-tdTomato-p10.536.1473-B3-3×P3_dsRed-B3-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; 20×UAS—20 repeats of the UAS upstream activating sequence; IVS—intervening sequence (intron); Syn21—synthetic promoter sequence; op1—operator sequence; jGCaMP7s-T2A-tdTomato-p10.536.1473—genetic construct designed for calcium imaging and fluorescent protein expression; B3—B3 integrase under heat shock control; 3×P3—three copies of the Pax-6 binding sites, serving as a promoter; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 23 is another example of a sequence for a vector of the pJAT series, which is referred to as pJAT71{dU6-3-attR3-Amp-ccdB-attR4-20×UAS-IVS-Syn21-op1-jGCaMP7s-T2A-tdTomato-p10.536.1473—B3-ie1-dsRed-B3-attR5-Chlor-SacB-attR6}. The abbreviations used to describe components of the sequence are commonly used in the art: dU6-3 U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; 20×UAS—20 repeats of the UAS upstream activating sequence; IVS—intervening sequence (intron); Syn21—synthetic promoter sequence; op1—operator sequence; jGCaMP7s-T2A-tdTomato-p10.536.1473—genetic construct designed for calcium imaging and fluorescent protein expression; B3—B3 integrase under heat shock control; ie1—ie1 promotor; dsRed—Discosoma sp. red fluorescent protein; Chlor—chloramphenicol resistance selection marker; SacB—sacB gene used as a negative selection marker. The components of the payload or donor DNA of the sequence are underlined.

SEQ ID NO: 24 is another example of a sequence for a vector of the pJAT series, which is referred to as pJAT74{dU6-3-attR3-Amp-ccdB-attR4-MCS-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; MCS—multiple cloning site; Chlor—chloramphenicol resistance selection marker; SacB—sacB gene used as a negative selection marker. The components of the payload or donor DNA of the sequence are underlined.

SEQ ID NO: 25 is another example of a sequence for a vector of the pJAT series, which is referred to as pJAT75{dU6-3-attR3-Amp-ccdB-attR4-ie1-EGFP,PhsFlp1-attR5-Chlor-SacB-attR6}. The abbreviations used to describe components of the sequence are commonly used in the art: dU6-3—U6 promoter that is derived from the third U6 gene in Drosophila melanogaster; attR3, attR4, attR5, and attR6—attR recombination recognition sequences; Amp—ampicillin resistance selection marker; ccdB—control of cell death B gene used as a negative selection marker; ie1—ie1 promotor; EGFP—enhanced green florescent protein; PhsFlp1—genetic construct that includes the FLP recombinase gene under the control of the heat shock promoter (Hsp70); Chlor—chloramphenicol resistance selection marker; SacB—sacB gene used as a negative selection marker. The components of the payload or donor DNA of the sequence are underlined.

SEQ ID NO: 26 is an example of a PCR primer used in accordance with the presently-disclosed subject matter, which is referred to herein as Tn5 ME B adaptor-attB4.

SEQ ID NO: 27 is an example of a PCR primer used in accordance with the presently-disclosed subject matter, which is referred to herein as Tn5 ME B adaptor-P767_attB5.

SEQ ID NO: 28 is an example of a PCR primer used in accordance with the presently-disclosed subject matter, which is referred to herein as Tn5 ME B adaptor-P776_attB5.

SEQ ID NO: 29 is an example of a PCR primer used in accordance with the presently-disclosed subject matter, which is referred to herein as Tn5 ME B adaptor-P777_attB5.

SEQ ID NO: 30 is an example of a PCR primer used in accordance with the presently-disclosed subject matter, which is referred to herein as Tn5 ME B adaptor-P795_attB5.

SEQ ID NO: 31 is an example of a PCR primer used in accordance with the presently-disclosed subject matter, which is referred to herein as Tn5 ME B adaptor-P832_attB5.

SEQ ID NO: 32 is an example of a PCR primer used in accordance with the presently-disclosed subject matter, which is referred to herein as Tn5 ME B adaptor-P859_attB5.

SEQ ID NO: 33 is an example of a PCR primer used in accordance with the presently-disclosed subject matter, which is referred to herein as Tn5 ME B adaptor-P887_attB5.

SEQ ID NO: 34 is an example of a sequence useful in connection with the presently-disclosed subject matter, which is referred to herein as attL3-tRNA-gRNA.

SEQ ID NO: 35 is an example of a sequence useful in connection with the presently-disclosed subject matter, which is referred to herein as attL4 (reversed).

SEQ ID NO: 36 is an example of a sequence useful in connection with the presently-disclosed subject matter, which is referred to herein as attL5.

SEQ ID NO: 37 is an example of a sequence useful in connection with the presently-disclosed subject matter, which is referred to herein as attL6 (reversed).

SEQ ID NO: 38 is an example of a sequence useful in connection with the presently-disclosed subject matter, which is referred to herein as L3-mel_attP2_HAL_L4.

SEQ ID NO: 39 is an example of a sequence useful in connection with the presently-disclosed subject matter, which is referred to herein as L5-mel_attP2_HAR_L6.

SEQ ID NO: 40 is an example of a sequence useful in connection with the presently-disclosed subject matter, which is Chlor-SacB Selectable Cassette. The abbreviations used to describe components of the sequence are commonly used in the art: Chlor—chloramphenicol resistance selection marker; SacB—sacB gene used as a negative selection marker.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes vectors and methods for that allow for simplified cloning and increased efficiency. Embodiments of the vectors and methods as disclosed herein can be used in connection with CRISPR/Cas-mediated homology directed repair (HDR).

The presently disclosed subject matter includes a cloning vector, which includes a first cassette having a nucleotide sequence comprising a first negative selection marker that is a ccdB gene, which is flanked by first non-identical attR recombination recognition sequences. In some embodiments, the cloning vector further includes a second cassette having a nucleotide sequence comprising a second negative selection marker that is not a ccdB gene, which is flanked by second non-identical attR recombination recognition sequences, wherein the first non-identical attR recombination recognition sequences are district from the second non-identical attR recombination recognition sequences.

The second negative selection marker of the cloning vector is a negative selection marker that is not a ccdB gene. As disclosed herein, the presently-disclosed subject matter is based, in part, on the discovery that, when attempting to build a dual-Gateway® plasmid following earlier examples, multiple copies of the standard Gateway® negative selection marker, ccdB, were found to be unstable. The second negative selection marker can be selected from negative selection markers known to those of ordinary skill in the art upon study of this document. In some embodiments, the second negative selection marker is a sacB gene. Additional examples of negative selection markers include, but are not limited to, rpsL, thyA, and upp.

In some embodiments, the cloning vector can include an additional selection marker. For example, in some embodiments the additional selection marker can be an antibiotic resistance marker. In some embodiments, the cloning vector can include a first antibiotic resistance marker in the first cassette, a second antibiotic resistance marker in the second cassette, and a third antibiotic resistance marker outside the first and second cassettes. The antibiotic resistance marker(s) can be selected from any antibiotic selection markers known to those of ordinary skill in the art upon study of this document. Examples of antibiotic selection markers include, but are not limited to, AmpR (ampicillin resistance), CamR (chloramphenicol resistance), and SmR (streptomycin resistance). Additional examples are set forth in a database of antibiotic resistant genes, which is accessible at the following site: card.mcmaster.ca/

In some embodiments, the cloning vector can include a promoter outside the cassettes. The promoter(s) can be selected from any promoter that drives RNA transcription known to those of ordinary skill in the art upon study of this document. In some embodiments, the promoter is a U6 promoter. Additional examples of promoters include, but are not limited to, CMV, EF1α, H1. Additional examples of PolIII promoters that could be used are identified by Ma (2014),²⁴ which article is incorporated herein by this reference. As will be appreciated by the skilled artisan, the promoter is selected with regard to a species of interest. As disclosed herein, in some embodiments, the species of interest is an insect species.

In some embodiments, the cloning vector further includes a multiple cloning site or any other DNA that is intended to be inserted into the genome by homology directed repair located between the cassettes, which is sometimes referred to herein as donor DNA or payload. In some embodiments, the donor DNA can include a sequence providing a germline source or ubiquitously expressed source of an enzyme for creating double-strand breaks. In some embodiments, the donor DNA can include attP landing sites. In some embodiments, the donor DNA can include marker genes. In some embodiments, the donor DNA can include a split-fluorescent protein system to report on simultaneous homology-directed repair (HDR).

The presently-disclosed subject matter further includes a donor vector, which includes a first homology arm sequence flanked by first non-identical attR recombination recognition sequences, and a short guide RNA (sgRNA) sequence adjacent the first or the second homology arm sequence and separated from the homology arm by an RNA polymerase termination sequence. Some embodiments of the donor vector further include a second homology arm sequence flanked by second non-identical attR recombination recognition sequences, wherein the first non-identical attR recombination recognition sequences are distinct from the second non-identical attR recombination recognition sequences.

As an alternative to using two homology arms, and as described herein, it is possible to introduce a single homology arm using the two outermost attR sites (e.g., attR3 and attR6 in examples provided herein). This would be desirable if, for example, the user wants to introduce a specific genomic change without any marker genes or effectors. In this regard, the donor DNA/payload need not be included. Genomic changes could be made, for example, through intentional design of the homology arm, e.g., to include one or more mutations.

Some embodiments of the donor vector further include a transfer RNA (tRNA) sequence or a pair of tRNA sequences flanking the sgRNA to provide precise splicing of sgRNA products from the RNA transcript. In some embodiments, the donor vector can include a tRNA sequence that is operably linked to the gRNA sequence. In some embodiments, tRNA sequences flank and are operably linked to the gRNA sequence. In some embodiments, the gRNA is a single-guide RNA (sgRNA), comprising a CRISPR RNA (crRNA) sequence and a trans-activating CRISPR RNA (tracrRNA) sequence. In some embodiments, the donor vector includes a gRNA region that comprises a first tRNA sequence, a crRNA sequence, a tracrRNA sequence, and a second tRNA sequence. In some embodiments, the gRNA region is downstream of a promoter.

In some embodiments, the donor vector can include a promoter, wherein the gRNA sequence is downstream of the promoter. The promoter(s) can be selected from any promoter that drives RNA transcription known to those of ordinary skill in the art upon study of this document. In some embodiments, the promoter is a U6 promoter. Additional examples of promoters include, but are not limited to, CMV, EF1α, H1. As will be appreciated by the skilled artisan, the promoter is selected with regard to a species of interest. As disclosed herein, in some embodiments, the species of interest is an insect species.

In some embodiments, the donor vector further includes a donor DNA/payload between the homology arms. In some embodiments, the donor DNA can include a sequence providing a germline source or ubiquitously expressed source of an enzyme for creating double-strand breaks. In some embodiments, the donor DNA can include attP landing sites. In some embodiments, the donor DNA can include marker genes. In some embodiments, the donor DNA can include a split-fluorescent protein system to report on simultaneous homology-directed repair (HDR).

In some embodiments, the cloning or donor vector of the presently-disclosed subject matter includes donor DNA/payload that includes a sequence providing a germline source or ubiquitously expressed source of an enzyme for creating double-strand breaks, attP landing sites, marker genes, or a split-fluorescent protein system to report on simultaneous homology-directed repair (HDR) of two plasmids. Examples of split-fluorescent protein systems include split green fluorescent protein (GFP), split yellow fluorescent protein (YFP), split red fluorescent protein (RFP), split cyan fluorescent protein (CFP), and split monomeric red fluorescent protein (mCherry).

It is contemplated that, in some embodiments, a germline source of Cas9, or other enzyme for creating double-strand breaks, will make use of germline-specific sequences for each species of interest. Alternatively, a ubiquitously expressed source can be provided, and can make use of a ubiquitous promoter from each species of interest, as will be appreciated by the skilled artisan, such as, for example from Actin gene. As will be appreciated by the skilled artisan, in addition to Cas9, there are other enzymes for creating double-strand breaks or DNA nicks. A non-exhaustive list of examples includes Cas12a/Cpf1, Cas13, and Cas12j/CasPhi. Additional examples include I-SceI endonuclease and transcription activator-like effector nucleases (TALENs).

With regard to donor DNA/payload, as will be appreciated by the skilled artisan upon study of this document, there are various payloads that can be used and can have some benefit, depending on the goals of the user. For example, payloads can be provided that generate in-frame protein fusions to generate tagged proteins that can be localized in situ and used to perform immuno-precipitation assays to identify protein targets through mass spectrometry or other assays.

Certain donor vectors of the presently-disclosed subject matter can include first and second homology arms. In some embodiments, the first homology arm sequence is operably connected to a gRNA region sequence.

In some embodiments, the cloning and donor vectors of the presently-disclosed subject matter can include donor DNA/payload.

In some embodiments, the vectors disclosed herein comprise a sequence selected from the sequence of any one of sequences as disclosed herein or a component thereof. For example, in some embodiments, the vectors comprise the sequence of SEQ ID NO: 40, which includes the components Chlor (chloramphenicol resistance selection marker) and SacB (sacB gene used as a negative selection marker) to provide a selectable cassette.

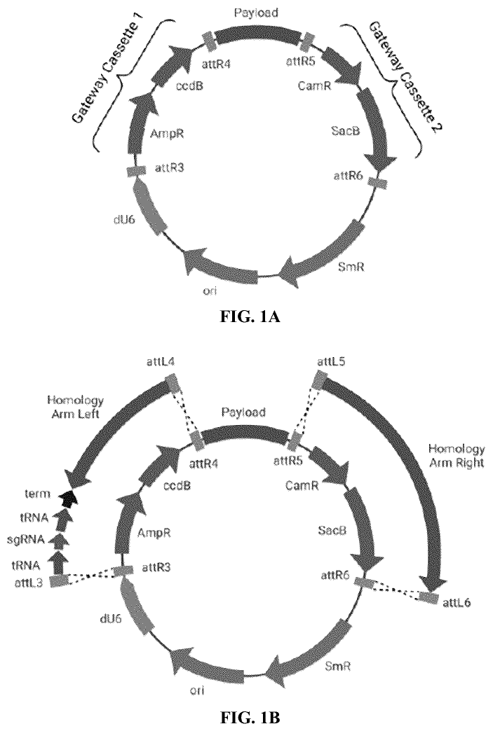

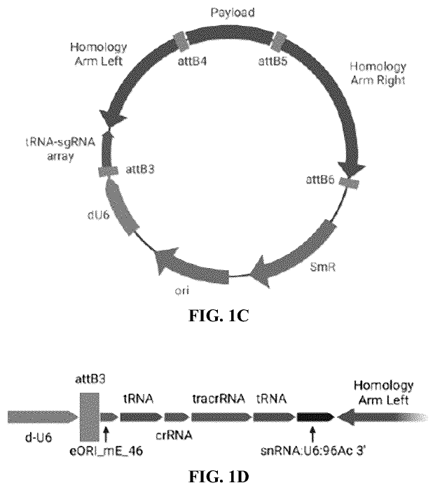

As depicted in herein, orientation of sequences of the homology arms can be “ends out.” In some embodiments, they can be “ends in.” The terms “ends out” and “ends in” refer to a specific orientation of the homology arm template with respect to the target site where a double-strand break (DSB) will be introduced. As depicted in FIG. 1B, in the “ends out” orientation, the ends of the homology arm template extend in opposite directions. By contrast, in the “ends in” orientation, the ends extend towards one another. Although both “ends in” and “ends out” orientations are contemplated within the scope of the presently-disclosed subject matter, in some embodiments there is a preference for the homology arms being in the same orientation as the genome sequence, i.e., “ends out.”

The presently-disclosed subject matter also includes a method of preparing a donor vector by simultaneously introducing two DNA fragments of interest into a cloning vector, which includes the steps of (a) providing the cloning vector as disclosed herein; (b) incubating the cloning vector with a first DNA fragment of interest flanked by attL recombination sites, a second DNA fragment of interest flanked by attL recombination sites, and a recombination enzyme; and (c) selecting a vector in which the first DNA fragment of interest has replaced the first cassette, and the second DNA fragment of interest has replaced the second cassette, thereby obtaining the donor vector.

In some embodiments of the method of preparing the donor vector, the first DNA fragment of interest comprises a first homology arm sequence and a gRNA region sequence, and the second DNA fragment of interest comprises a second homology arm sequence. The efficiency of cloning homology arms into the vectors, as disclosed herein, is greater than about 95%. As will be appreciated by the skilled artisan, in some embodiments, transformed colonies are picked after cloning, grown in with appropriate selection media (e.g., antibiotics) to ensure retention of the desired vector, and DNA can be purified and sequenced from these cultures.

The presently-disclosed subject matter also includes a method of integrating a donor DNA into a genome of a cell, which includes the steps of introducing a donor vector as disclosed herein into a cell; and providing to the cell a source of an enzyme for creating double-strand breaks (DSBs) or DNA nicks guided by the sgRNA encoded in the donor vector.

As disclosed herein, it was unexpectedly and surprisingly discovered that the unique vectors as described herein yielded far higher HDR integration efficiencies on average than has been observed others using other methods. In this regard, in some embodiments, the method of integrating a donor DNA into a genome of a cell also involves the step of confirming an efficiency of integration of greater than about 5, 10, 15, 20, 25, 30, 35, 40, 45, or 50%.

In some embodiments, in the method of integrating a donor DNA into a genome of a cell the enzyme for creating DSBs is provided to the cell by: injection of the enzyme as a protein; injection of mRNA encoding the enzyme; and/or germline expression of a transgenic source of the enzyme. In some embodiments, the germline expression of the transgenic source is provided using a second donor vector, wherein the donor DNA comprises a nucleotide sequence encoding the enzyme.

In some embodiments of the method, in which multiple donor vectors as disclosed herein are injected into the cell, each donor vector has a distinct donor DNA.

In some embodiments, in the method of integrating a donor DNA into a genome of a cell, the cell is in an animal or plant embryo. In some embodiments of the method, the animal is an insect.

Insects of interest to the presently-disclosed subject matter include, but are not limited to, vinegar flies, including species of the Drosophila genus, including melanogaster and non-melanogaster Drosophila; aphids, including any species that feed on domesticated crops, for example, including species of the genera Acyrthosiphon, Aphis, Brevicoryne, Cerataphis, Diuraphis, Eriosoma, Macrosiphum, Melanaphis, Myzus, Rhopalosiphon, Sitobion, and Toxoptera; and mosquitoes that attack humans and domesticated animals, including species of the genera Aedes, Anoheles, Culex, Mansonia, and Culliseta.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, Addgene and GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as Addgene and GenBank. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “ccdB” or “ccdB gene” refers to the control of cell death B gene, which is a part of the control of cell death (ccd) operon found in plasmids of bacteria. As will be appreciated by one of ordinary skill in the art, the ccdB gene can be used in cloning vectors as a negative selection marker.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used herein, the terms “donor DNA” and “payload” are used interchangeably to refer to DNA that is intended to be inserted into a genome by homology directed repair (HDR).

The term “homology arm” refers to a DNA sequence that shares sequence similarity or homology with a target sequence in the genome. As known to those of ordinary skill in the art, homology arms can be designed to flank a DNA sequence of interest, with one homology arm located upstream (5′ end) and the other downstream (3′ end) of the target sequence. These homology arms enable the precise integration of the introduced DNA into the specific genomic locus through the process of recombination.

The term “marker gene” refers to a sequence introduced to indicate the presence of specific genetic material or to monitor certain biological processes. They are often used to identify successfully transformed cells or to study gene expression.

A “multiple cloning site” is a specific region within a cloning vector that contains multiple unique restriction enzyme recognition sequences. It serves as a location for the insertion of DNA fragments during the cloning process.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

A “promoter” is a DNA sequence that plays a role in initiating and regulating the transcription of a gene. Promoters are typically located upstream of the gene they control, and facilitate the initiation of transcription by providing a recognition site for RNA polymerase and associated transcription factors.

A “recombination recognition sequence,” also known as a recombination site or recognition site, refers to a specific DNA sequence that serves as a recognition site for recombination enzymes. Recombination recognition sequences are useful for facilitating site-specific DNA recombination events, such as integration, excision, inversion, or rearrangement of DNA segments. Examples of categories of different recombination recognition sequences include attB, attP, attL, and attR. There are various examples within each category. For example, attB1, attB2, attB3, attB4, attB5, and attB6 are all different examples of attB recombination recognition sequences. For another example, attP1, attP2, attP3, attP4, attP5, and attP6 are all different examples of attP recombination recognition sequences. For another example, attL1, attL2, attL3, attL4, attL5, and attL6 are all different examples of attL recombination recognition sequences. For another example, attR1, attR2, attR3, attR4, attR5, and attR6 are all different examples of attR recombination recognition sequences. In this regard, where non-identical attR recombination recognition sequences are useful, the can be selected from such distinct examples. Non-identical attR recombination recognition sequences could be, for example, attR1 and attR2, attR1 and attR3, attR1 and attR4, attR1 and attR5, attR1, and attR6, attR2 and attR3, attR2 and attR4, attR2 and attR5, attR2 and attR6, attR3 and attR4, attR3 and attR5, attR3 and attR6, attR4 and attR5, attR4 and attR6, and attR5 and attR6,

A “selection marker” refers to a gene or DNA sequence that confers a desired selective advantage. For example, it can be used to identify and select cells or organisms that have successfully incorporated a desired DNA fragment or undergone a specific genetic modification. Selection markers can be included in cloning vectors or donor vectors to enable the identification and enrichment of cells that have taken up the vector or undergone the desired genetic manipulation. A common type of selection marker is an antibiotic resistance marker, which encode proteins that confer resistance to specific antibiotics, such as Spectinomycin. When cells or organisms are grown in the presence of the corresponding antibiotic, only those that have incorporated the selection marker will survive and propagate, while the non-transformed or non-modified cells will be eliminated. A “negative selection marker” refers to a gene or DNA sequence that confers a lethality to cells that carry it. It is used as a means to selectively eliminate or counterselect against cells that have retained the marker or undergone certain genetic modifications. Examples of negative selection markers include, for example, ccdB, sacB, rpsL, thyA, and upp.

A “termination sequence” is a DNA sequence that signals the termination or completion of transcription during gene expression. It is also known as a transcription termination signal or terminator. Termination sequences are typically located downstream of the coding region of a gene and serve as recognition sites for the termination of RNA synthesis by RNA polymerase.

The terms “vector” and “plasmid” are used interchangeably herein to refer to a replicable nucleic acid molecule from which one or more heterologous proteins can be expressed when the vector is transformed into an appropriate host cell. Reference to a vector includes those vectors into which a nucleic acid molecule encoding a polypeptide or fragment thereof can be introduced, typically by restriction digest and ligation. Reference to a vector also includes those vectors that contain nucleic acid molecule encoding a polypeptide. The vector is used to introduce the nucleic acid molecule encoding the polypeptide into the host cell for amplification of the nucleic acid molecule or for expression/display of the polypeptide encoded by the nucleic acid molecule. The vectors can remain episomal, or can be designed to effect integration of a gene or portion thereof into a chromosome of the genome.

The term “vector” is inclusive of an expression vector, includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors can be derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA.

In some cases a vector can be referred to as a cloning vector, which is a DNA molecule used for the cloning and propagation of certain DNA fragments. In some cases a vector can be referred to as a donor vector, which is a vector used to supply a DNA fragment for integration or modification with a genome of a cell.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: The pJAT Series Dual-Gateway® Cloning Platform

A plasmid platform was built that could utilize recent advances in DNA synthesis technology to simplify the cloning of reagents for CRISPR-Cas9 induced HDR. It is now possible to synthesize long dsDNA fragments containing repetitive DNA attL sequences at the ends at a reasonable price (for example, 7 cents per bp from Twist Bioscience). In view of the availability of these long dsDNA fragments, as disclosed herein, a dual-Gateway® cloning platform for direct cloning of two fragments was designed (FIG. 1A-C). To further reduce the number of plasmids that were required for experiments, a tRNA-gRNA-tRNA design⁵ was incorporated directly into the synthesized fragments (FIG. 1B, D).

Initial attempts at building a dual-Gateway® plasmid followed earlier examples and simply duplicated the standard ccdB negative selection gene in two separate attR-attR cassettes⁸. However, when using this approach, every plasmid recovered during cloning carried a mutation in one of the ccdB genes, suggesting that high ccdB dosage is deleterious to E. coli⁴. By the invention disclosed herein, this issue was favorably overcome. In particular, as disclosed herein, one of the ccdB genes was cleverly replaced with a sacB gene. The sacB gene allows negative selection by growing E. coli on plates containing high levels of sucrose⁹.

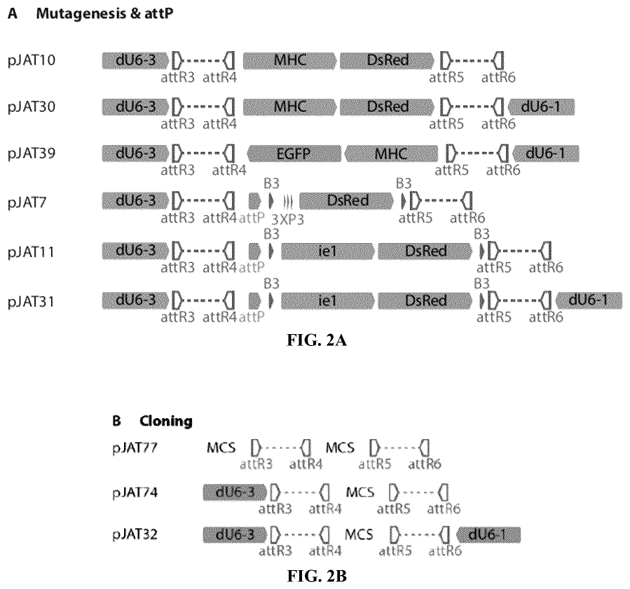

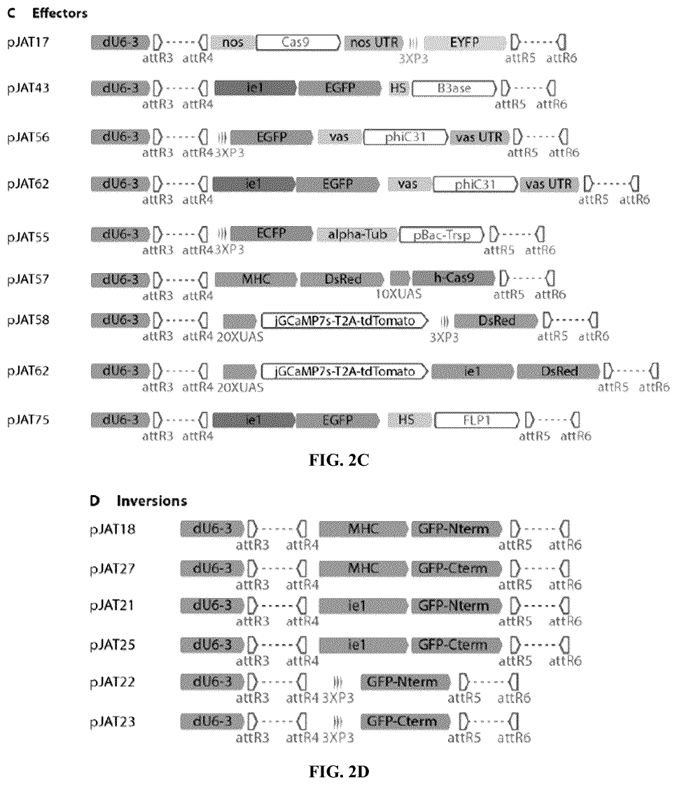

Embodiments of the pJAT plasmids described in these examples contain one or two U6 promoters flanking the Gateway® cloning cassettes or restriction sites that can be used to introduce new U6 promoters. pJAT plasmids containing multiple kinds of payloads have been generated, including, for example, (1) a germline source of Cas9, (2) attP landing sites, (3) marker genes, and (4) a split-GFP system to report on simultaneous HDR of two plasmids (FIG. 2). All of the current pJAT plasmids utilize fluorescent reporter genes expressed in different anatomical regions¹⁰, which allow recombination of multiple reagents into a single wild-type fly without the use of balancer chromosomes (chromosomes carrying multiple inverted regions that prevent recombination between homologous chromosomes). To facilitate introduction of novel payloads, one pJAT plasmid has been generated that contains a multiple cloning site for both standard ligation based cloning and Golden Gate cloning. (FIG. 2).

For plasmid construction, homology arms are synthesized with compatible attL sites and the company's adaptor sequences are left on, both because this reduces synthesis cost and because the adaptor sequences do not conflict with Gateway® cloning. Synthesis of arms confers many benefits compared with PCR based construction of HDR plasmids, including the ability to include exogenous sequences, such as the tRNA-gRNA-tRNA array and other elements, as described further below, and the ability to introduce single base pair modifications. Cloning requires standard Gateway® cloning reagents and little hands-on time. An example of a full protocol describing design of homology arms and all steps of cloning is provided in Example 10.

### Example 2: pJAT Plasmids Yield High Efficiency HDR

Initial experiments performed with the pJAT plasmids resulted in unexpected and surprising results. In particular, these experiments with pJAT plasmids yielded surprisingly high efficiencies of HDR of approximately 40-50%. Such high efficiencies of HDR in Drosophila have only rarely been observed previously. Accordingly, many loci in D. melanogaster were systematically targeted and it was discovered that all loci could be successfully targeted.

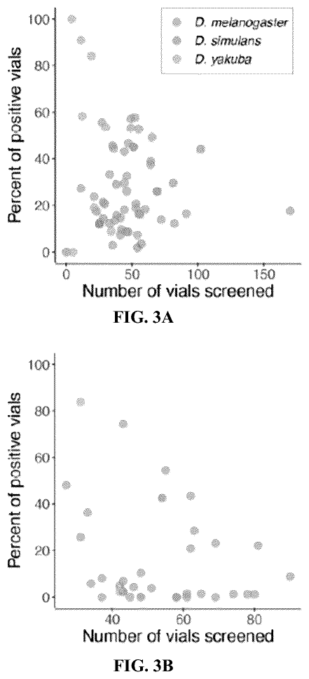

Confirmation was obtained that all HDR products were integrated into the correct locations using TagMap¹¹ with pJAT-specific primers (Materials and Methods). The average integration efficiency—that is, the fraction of fertile GO injected eggs that yielded at least one positive event in the next generation—was approximately 25% for insertions into genes in which null mutations are homozygous viable and was more variable and lower on average for genes in which null mutations were homozygous lethal (FIG. 3).

Seven of the injections into lethal genes yielded no events, but these were repeated injections using modified pJAT plasmids into only two loci. Despite these failures, other injections into these two loci yielded some events, though at low frequency. Thus, all genes were targetable with pJAT plasmids, but a few genes, and specifically a subset of those that are homozygous lethal, showed low efficiency. This low efficiency did not appear to result from the specific gRNA sequences used (data not shown) or the homology arm lengths (Table 1). One possibility, which requires further investigation, is that the high efficiency of pJAT plasmids may induce homozygous null genotypes in many cells of the developing embryo, leading to lethality or sterility.

TABLE 1
Test of effect of homology arm lengths on efficiency of HDR.
Increasing homology arm lengths for l(2)gl from 250 bp to 1000
bp did not increase HDR efficiency. For Ir41a, homology arms
longer than 250 displayed the highest HDR efficiency.

| | Left | Right | | | | |
|---|---|---|---|---|---|---|
| | Homology | Homology | | | | Effi- |
| Targeted | Arm | Arm | Number | Number | Number | ciency |
| Gene | Length | Length | Injected | Fertile | positive | (%) |
| l(2)gl | 250 | 250 | 265 | 65 | 1 | 2 |
| l(2)gl | 250 | 1000 | 240 | 48 | 0 | 0 |
| l(2)gl | 1000 | 1000 | 265 | 45 | 0 | 0 |
| Ir41a | 60 | 125 | 300 | 35 | 1 | 3 |
| Ir41a | 120 | 125 | 250 | 46 | 4 | 9 |
| Ir41a | 250 | 250 | 260 | 64 | 25 | 39 |
| Ir41a | 600 | 600 | 260 | 46 | 12 | 26 |

### Example 3: Multiple Features Contribute to High Efficiency of pJAT Plasmids

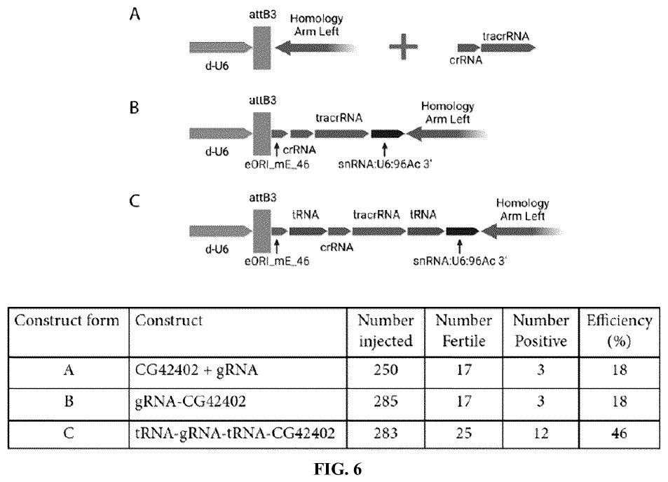

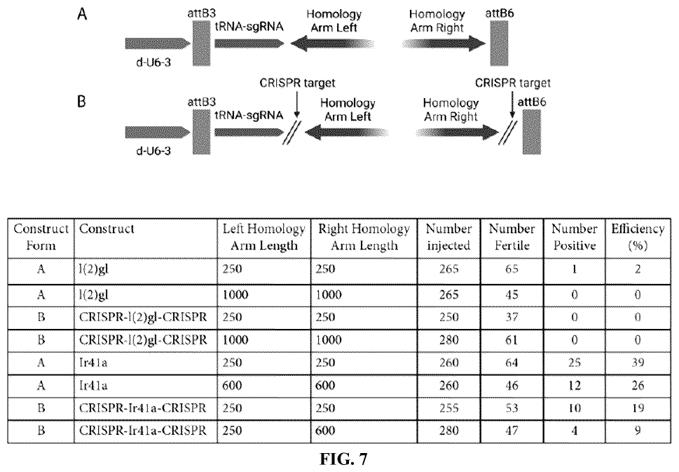

To determine possible reasons for the high efficiency of HDR with pJAT plasmids, sections of the synthesized fragments were systematically varied. Two features were identified that improved HDR efficiency. First, as has been documented previously⁵, flanking the sgRNA with tRNAs increased efficiency over co-injection with a sgRNA (FIG. 6). Surprisingly, a pJAT plasmid containing the sgRNA, but no flanking tRNAs, had similar efficiency to co-injecting with prepared sgRNA (FIG. 6), indicating that Cas9 can utilize a sgRNA even when it is embedded within flanking irrelevant RNA sequences, similar to results observed by others⁷. Second, use of intact dsDNA plasmids yielded higher efficiency HDR than plasmids that contained homology arms flanked by CRISPR-Cas9 cut sites (FIG. 7), which should linearize the plasmid DNA in vivo and which has been recommended previously¹². It is possible that the relatively long homology arms used here improved efficiency, but it was discovered that arms as short as 250 bp yielded similar efficiency as longer arms (Table 1). Shorter arms were not tested, since it is challenging to synthesize short arms with the long repeated attR sequences on both ends. The concentration of sgRNA produced from a single U6 promoter does not appear to be limiting, since synthesis of the same gRNA from two U6 promoters did not increase HDR efficiency (FIG. 8).

While pJAT plasmids provide sufficient gRNA for efficient HDR, it was contemplated that the quantity of Cas9 protein may be limiting when Cas9 is introduced exogenously, either as mRNA or protein. To test this idea, a pJAT plasmid was built that drives expression of a Drosophila codon optimized Cas9 mRNA from a germline specific nanos promoter with a nanos 3′UTR to localize Cas9 mRNA to the developing oocytes (FIG. 2) and found that it increased HDR efficiency approximately 2-3 fold compared to co-injection with Cas9 mRNA (Table 2).

TABLE 2
Effect of germline Cas9 on HDR efficiency. Injection of pJAT plasmids
into embryos expressing a germline source of Cas9 (from nanos-Cas9)
exhibited considerably higher HDR efficiency than co-injection of pJAT
plasmids with Cas9 mRNA.

| | | Number | Number | Number | Efficiency |
|---|---|---|---|---|---|
| Cas9 source | Targeted gene | injected | Fertile | Positive | (%) |
| germline | D. melanogaster | 240 | 33 | 11 | 33 |
| | CG42402 | | | | |
| mRNA | D. simulans | 280 | 52 | 8 | 15 |
| | CG42402 | | | | |
| germline | D. simulans | 240 | 81 | 24 | 30 |
| | CG42402 | | | | |
| germline | D. melanogaster | 250 | 102 | 45 | 44 |
| | Osi2 | | | | |
| mRNA | D. simulans Osi2 | 240 | 54 | 6 | 9 |
| mRNA | D. simulans Osi2 | 250 | 102 | 14 | 14 |
| germline | D. simulans Osi2 | 260 | 102 | 45 | 44 |

All of the experiments described above were performed by incubating injected embryos at 22° C. after injection. Given the known temperature sensitivity of Cas9¹³, a small test of incubation temperature was performed by placing eggs at either 20° C., 22° C., or 25° C. for 4 h after injection. An increase in integration efficiency was observed at higher temperatures (Table 3), suggesting that the effect of temperature on CRISPR-Cas9 HDR in Drosophila should be investigated further.

TABLE 3
Plasmid p{dU6-3-mel_attp2-HAL-B3-ieldsRed-B3-mel_att2-HAR} was
injected into pre-blastoderm embryos of Oregon-R nanos-Cas9 line 72A
and embryos were subsequently transferred to one of three temperatures
for four hours. Surviving adults were crossed to Oregon-R and offspring
were screened for presence of iel-DsRed.

| 4 hour | Number | Number | Number | Number | Efficiency |
|---|---|---|---|---|---|
| incubation | larvae | viable | fertile | positive | (%) |
| 20° C. | 54 | 40 | 26 | 5 | 19 |
| 22° C. | 57 | 41 | 15 | 4 | 27 |
| 25° C. | 56 | 38 | 14 | 8 | 57 |

Note that the efficiencies reported for many experiments shown in FIG. 3 resulted from injections using Cas9 mRNA and with incubation at 22° C., which were subsequently discovered to be sub-optimal conditions. Thus, the average efficiencies reported here may underestimate the average efficiency using optimal conditions. The availability of a high-efficiency CRISPR-HDR platform that works in multiple species invites new kinds of experiments. Next, two kinds of experiments are illustrated, which have been challenging to implement with previous technology.

### Example 4: Scarless Mutagenesis with pJAT Plasmids

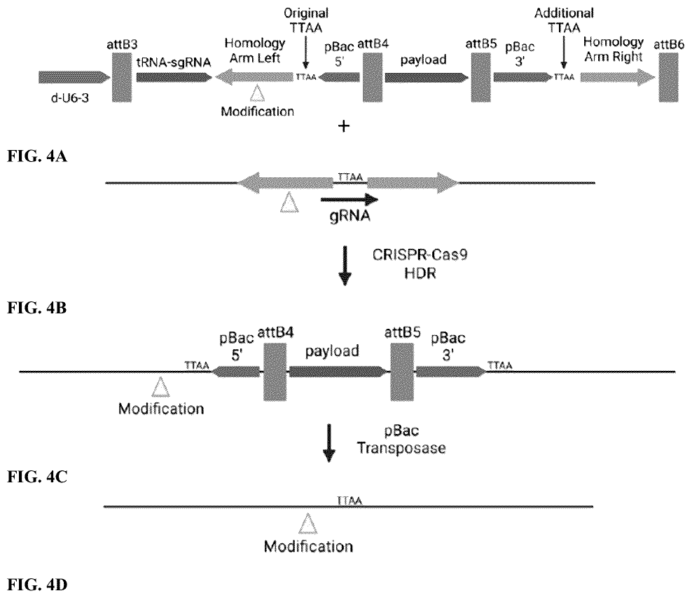

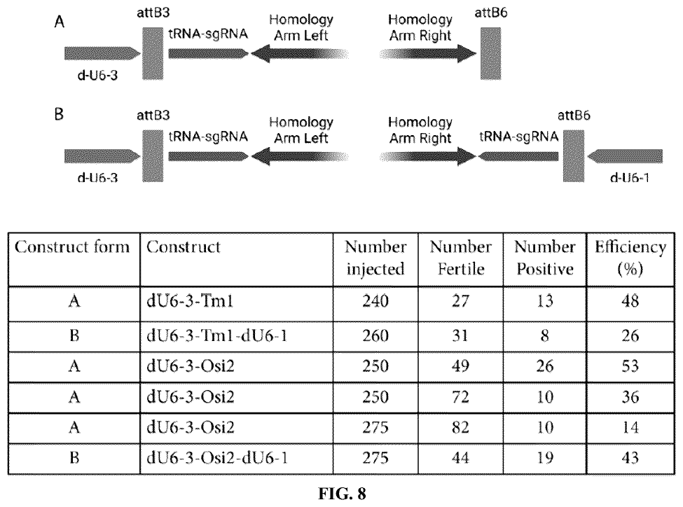

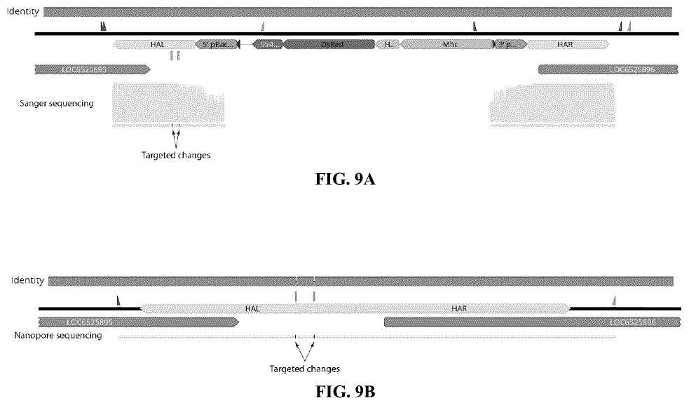

The use of synthesized homology arms and Gateway® cloning can simplify the performance of scarless mutagenesis. One approach, would be to insert a selectable marker at a target genome location and then, in a second step, to replace the selectable marker with the intended genomic changes¹⁴. A second approach, which is illustrated here, is to place a selectable fluorescent marker between piggyBac transposon arms, which are included in the synthesized DNA fragments adjacent to homology arms that include the intended genomic changes¹⁵. It was discovered that variable tract lengths are integrated into the genome and it is therefore best to place the intended genomic changes close to the pBac arms and to sequence the target region in HDR integrants to identify flies that carry the intended changes. The selectable marker, together with the piggyBac arms, is then removed by crossing flies to a strain expressing the piggyBac transposase gene (FIG. 4). In the experiment performed here, 58% integration efficiency was observed (7 of 12 fertile injected GO yielded integrants) into a site in D. yakuba and the pBac sequence was efficiently removed from multiple lines. This approach allowed efficient recovery of two scarless individual nucleotide changes at the targeted locus (FIG. 9A-9B).

### Example 5: pJAT Plasmids can Direct One-Step Targeted Chromosomal Inversions

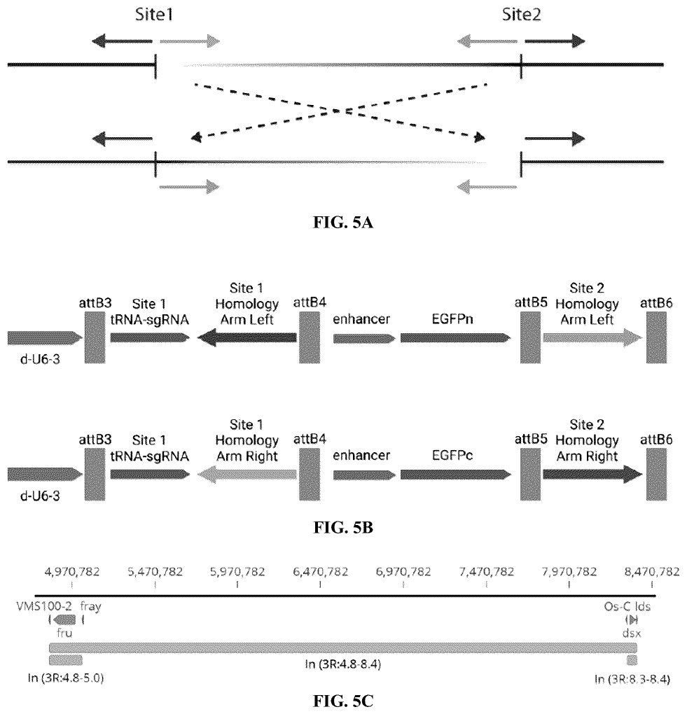

The HDR efficiency of pJAT plasmids was considerably higher than has been observed using other approaches and this high efficiency emboldened attempts at more complex experiments. One experiment that would be of considerable value, especially for studies of non-melanogaster Drosophila species would be the ability to generate chromosomal inversions, which could subsequently be used to balance deleterious alleles¹⁶. This experiment requires efficient cutting at two chromosomal sites followed by inversion of the intervening sequence (FIG. 5A). To encourage production of the inversion event, a pair of pJAT plasmids were designed where each plasmid carried homology arms oriented in the same direction as the intended inverted chromosomal region (FIG. 5B). To simplify detection of simultaneous integration of both plasmids, potentially indicative of an inversion, three pairs of pJAT plasmids were built, expressing split-GFP in either the eyes, the thorax, or the abdomen (FIG. 2).

Three inversions of approximately 58 kb to 3 Mbp were targeted in Drosophila yakuba to provide potential balancer chromosomes for reagents that had previously been built at the fruitless and doublesex genes in this species (FIG. 5C). Breaks within genes that are expected to be homozygous deleterious or lethal were targeted. Multiple events were detected for each targeted inversion, at efficiencies of 8-23% (Table 4).

TABLE 4
Efficiency of targeted inversion using pairs of pJAT plasmids. Plasmids
were submitted to two companies for injection into D. yakuba flies
carrying a nos-Cas9 transgene. Inversion efficiency was similar for the
two companies and for inversions of different distances from
58-3,543 kbp.

| | Injection | Distance | | | Efficiency |
|---|---|---|---|---|---|
| Inversion | Company | (kbp) | Screened | Positive | (%) |
| In(VMS-fray) | Rainbow | 200 | 17 | 2 | 12 |
| In(OsC-Ids) | Rainbow | 58 | 14 | 2 | 14 |
| In(VMS-Ids) | Rainbow | 3543 | 12 | 1 | 8 |
| In(VMS-fray) | Genetivision | 200 | 12 | 1 | 8 |
| In(OsC-Ids) | Genetivision | 58 | 26 | 6 | 23 |
| In(VMS-Ids) | Genetivision | 3543 | 32 | 5 | 16 |

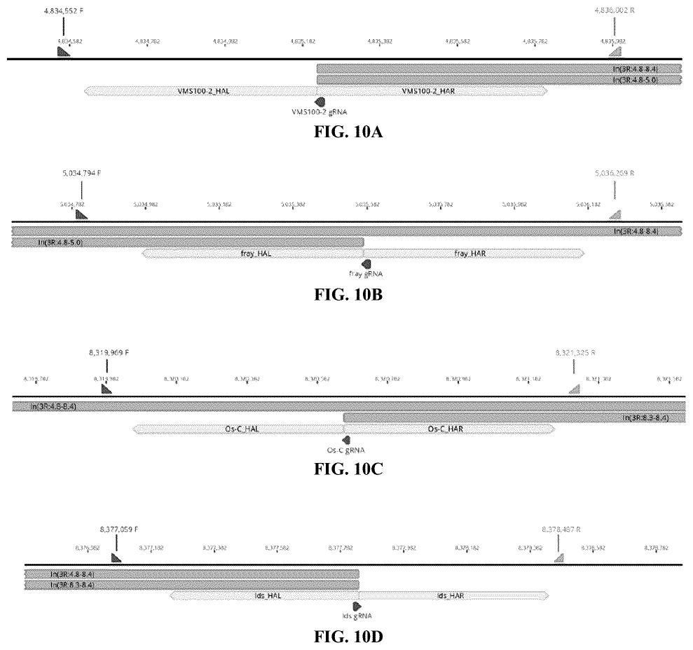

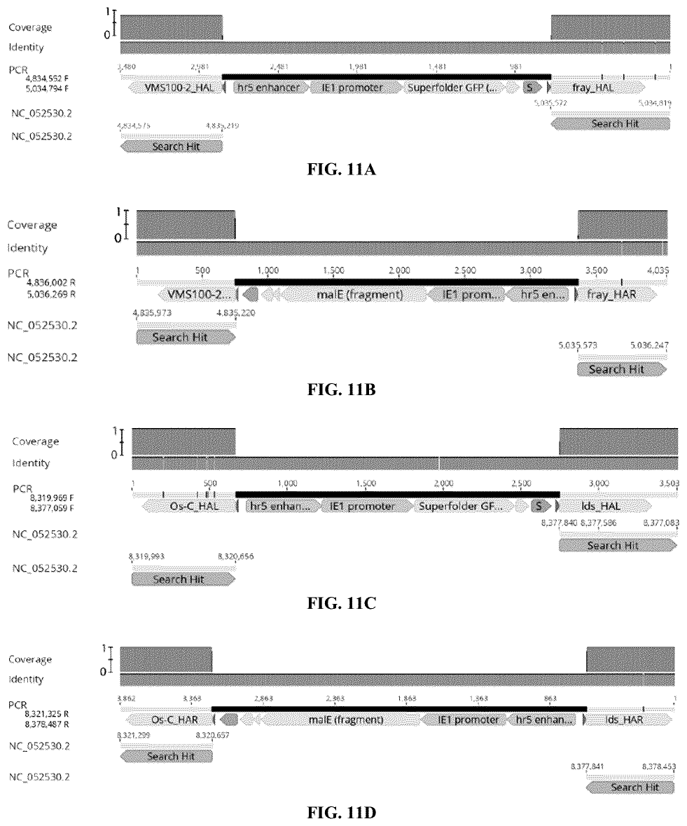

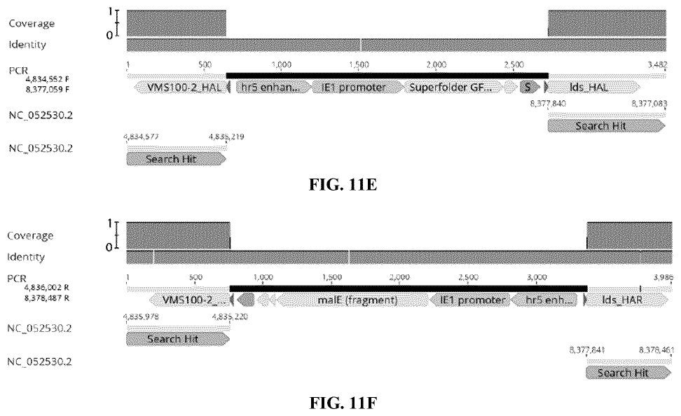

Fragments corresponding to the expected inversion event breakpoints were PCR amplified (FIGS. 10A-10D) and it is confirmed that the sequences of both breakpoints were oriented as expected (FIGS. 11A-11F). The three D. yakuba inversions were crossed to null alleles of either dsx or fru, which they were designed to balance, and it was discovered that they all balanced the targeted alleles for multiple generations. Thus, single inversions can be quickly introduced to balance specific alleles in Drosophila species. This technology will substantially simplify the maintenance of transgenic reagents and mutants in non-melanogaster Drosophila species. It is possible that multiple inversions can be introduced serially onto a single chromosome arm to generate balancer chromosomes of even greater utility¹⁰, and three sets of split-GFP reagents are provided, which may enable these experiments (FIG. 2).

### Discussion of Examples 1-5

A series of unique plasmids has been developed, which provide a simple cloning platform and enable high efficiency HDR in multiple Drosophila species. While Drosophila have been used in these examples, the plasmids and methods could be used in other insects, as described herein. The plasmid design is flexible and allows introduction of any payload of interest. The efficiency of pJAT plasmids is so high that it is almost certainly preferable to random integration via transposable elements for most purposes and may even be more efficient than the most widely used method of transgenesis in Drosophila at the moment, site-specific recombination into attP sites¹⁷. It has been reported previously that some strains and some species appear to be resistant to specific transposable elements and to attP-attB integration¹⁸. In fact, multiple transgenic approaches were attempted to introduce a nos-Cas9 transgene into the specific D. simulans strain used in this study without success, but multiple independent events were identified with a single injection of a pJAT plasmid carrying nos-Cas9. Thus, pJAT plasmids appear to be a preferable method of transgenesis to random integration using transposable elements, especially for studies of non-melanogaster Drosophila species.

The pJAT plasmids were designed for ease of cloning using synthesized homology arms that incorporate tRNA-sgRNA-tRNA arrays. Because these arms are synthesized, any modifications to the native sequence can be introduced during synthesis and then integrated into the genome. The power of this approach is illustrated by including mobilization sequences of the piggyBac transposon into the synthesized arms. This allows efficient scarless removal of the transposon and reporter gene, leaving behind substitutions introduced into the genome (FIG. 4). An additional illustration of the utility of this approach is that additional CRISPR target sites can be introduced flanking the homology arms, allowing the homology arms and payload to be linearized in vivo (FIG. 7). While this does not appear to increase HDR efficiency using pJAT plasmids in Drosophila (FIG. 7), consistent with reports of HDR induced by zinc-finger nucleases in Drosophila showing that circular donor DNA was more efficient than linearized DNA², linearization may increase HDR efficiency in other systems¹⁹.

Initial attempts were directed to building a double-Gateway® system using multiple ccdB containing cassettes, as has been demonstrated by others⁸. However, it was discovered during subsequent cloning that in every case one of the two ccdB genes had been inactivated. This may result from the dosage sensitivity of E. coli to ccdB copy number⁴. Therefore, a unique Gateway®-like second cloning cassette was developed, which cleverly makes use of a SacB gene, SacB genes have strong negative selective capabilities²⁰. When E. coli are grown on high levels of sucrose, expression of the SacB gene causes lethality. Thus, this stabilized double-Gateway®-like system dramatically improved cloning efficiency of two synthesized fragments containing complementary attL sites over a design that utilizes two cassettes both using the ccdB gene.

While the Janelia Atalanta series was designed to simplify cloning, it was unexpected and surprising to discover that it also increases HDR efficiency, compared with other methods that have been tested and reported, which is usually about 1-10% on average and where often HDR in many genes fails^{3,6}. Although difficult to quantify, publication bias may mean these reported efficiencies over-estimate true efficiencies, since there are multiple examples of failed CRISPR-HDR attempts in Drosophila species performed by other research groups that have been made known to the present inventor.

So far, every locus attempted has been able to be targeted and on average targeting genes or genomic regions that have non-lethal mutant phenotypes results in approximately 25% of fertile injected animals yielding correct insertion events. A series of experiments were performed to try to determine what might contribute to this increased efficiency and it appears that multiple factors contribute to higher efficiency HDR. Some of the most important factors appear to be (1) production of sgRNA from a tRNA-sgRNA-tRNA array, (2) injection into embryos with a germline source of Cas9 protein, (3) homology arms of at least 250 bp long, (4) circular plasmid DNA, and possibly (5) increasing the incubation temperature to 25° C. for a time after injection.

Despite the overall high efficiency of HDR with pJAT plasmids, a strikingly lower efficiency on average was identified with plasmids targeting genes with lethal null phenotypes. Even more surprising, a subset of these “lethal” genes showed high efficiencies. In limited experiments, no evidence was found that these lower efficiencies resulted from the specific gRNA or homology arms used. One possibility is that the pJAT plasmids are so efficient that when the reagents work at one targeted allele in an individual, the second allele is also hit at high frequency. This would cause injected animals to be mosaic for homozygous null mutations, which may lead to lethality of sterility of many flies carrying HDR events.

The most powerful demonstration of the high efficiency of pJAT plasmids is that they could be used to introduce two simultaneous breaks in a single chromosome and facilitate inversion of large chromosomal regions through HDR (FIGS. 5, 10A-10B, and 11A-11D). Surprisingly, three independent experiments, using two separate injection companies, yielded high efficiency inversions (Table 4). These inversions provide practical benefits, since they can be used to balance deleterious alleles and will simplify stock maintenance. It may also be possible to use pJAT plasmids to generate multiply inverted balancer chromosomes with wide applicability¹⁶. It has not escaped attention that this approach may also allow reversion of naturally occurring chromosomal inversions²¹, which often become hot-spots of evolutionary divergence²². Such experiments may allow, for the first time, detailed recombination-based identification of loci “locked” within non-recombining inversions that have contributed to phenotypic divergence.

The only species-specific component of the pJAT plasmids is the U6 promoter, and this promoter can be easily swapped with a different species-specific or inducible promoter. It is possible that pJAT plasmids may provide an improved CRISPR-HDR platform for other species. Therefore pJAT77 (SEQ ID NO: 5) was generated, where cloning sites can allow any promoter to be placed on either or both sides of the Gateway® cloning cassettes.

### Example 6: Construction of Embodiments of Janelia Atalanta (pJAT) Plasmids

pJAT plasmids were assembled by Golden Gate cloning²³ of PCR amplified and synthesized fragments. Several examples of plasmid sequences of the presently-disclosed subject matter are provided as SEQ ID NO: 1-25.

Gateway® compatible homology arms were synthesized by Twist Bioscience with Adaptors On. Because these sequences contain almost identical attL sites at either end, synthesis is inefficient and often fails with Adaptors Off. In addition, since these fragments are cloned into the pJAT plasmids using Golden Gate cloning, there is no need to remove the Twist synthesis adaptors. Golden Gate cloning was performed as follows.

| | |
|---|---|
| pJAT plasmid (~50 ng/uL) | 1 uL |
| Homology arm left (50 ng/uL) | 0.5 uL |
| Homology arm right(50 ng/uL) | 0.5 uL |
| LR Clonase II | 0.25 uL |

Reaction was incubated at 25° C. in a thermocycler for at least several hours. Then 0.125 uL of Proteinase K (provided with LR clonase kit) was added and sample was incubated in a thermocycler at 37° C. for 20 minutes. The entire reaction volume was gently mixed with at least 25 μL of Zymo Mix&Go Competent Cells thawed on ice. The mixture was incubated for 30 minutes on ice, heat shocked for 30 seconds at 42° C., and returned to ice. 100 μL of SOC was added and the mixture was shaken at 200 RPM at 37° C. for 1 hour. The entire mixture was plated on Tryptone-Yeast+Sucrose plates+Spectinomycin plates. The entire protocol, including a guide for designing and ordering homology arms, is provided at Protocols.io.

### Example 7: Injection of Drosophila Embryos

pJAT plasmids were injected into Drosophila embryos by Rainbow Transgenics or Genetivision. Initially, plasmids were co-injected with Cas9 mRNA generated by in vitro transcription from plasmid encoding Cas9¹⁰. After fly lines carrying a nanos-Cas9 transgene (pJAT17 (SEQ ID NO: 1)) were generated, plasmids were injected into flies homozygous for pJAT17 (SEQ ID NO: 1) and fertile adults emerging from injected eggs were outcrossed to a wild-type strain and offspring were screened for fluorescent reporter genes. The genomic insertion location of all integrants were determined using TagMap¹¹ with the following TagMap PCR primers, where the underlined sequence represents the Tn5 MEB adaptor and the non-underlined sequence represents the sequence specific to each pJAT plasmid.

| Primer: | Sequence: |
|---|---|
| Tn5 ME B | GTCTCGTGGGCTCGGAGATGTGTATAAGAGACAGAGCTGGACAACTTTGTATAGAAAAG |
| adaptor-attB4 | (SEQ ID NO: 26) |
| Tn5 ME B | GTCTCGTGGGCTCGGAGATGTGTATAAGAGACAGCGTCTCACAACTTTGTATACAAAAG |
| adaptor- | (SEQ ID NO: 27) |
| P767_attB5 | |
| Tn5 ME B | GTCTCGTGGGCTCGGAGATGTGTATAAGAGACAGCGAGTATAATCAACTTTGTATACAAAAG |
| adaptor- | (SEQ ID NO: 28) |
| P776_attB5 | |
| Tn5 ME B | GTCTCGTGGGCTCGGAGATGTGTATAAGAGACAGAAGCAACCCAACTTTGTATACAAAAG |
| adaptor- | (SEQ ID NO: 29) |
| P777_attB5 | |
| Tn5 ME B | GTCTCGTGGGCTCGGAGATGTGTATAAGAGACAGAACAACTAGTCAACTTTGTATACAAAAG |
| adaptor- | (SEQ ID NO: 30) |
| P795_attB5 | |
| Tn5 ME B | GTCTCGTGGGCTCGGAGATGTGTATAAGAGACAGCTCTCTAGTCAACTTTGTATACAAAAG |
| adaptor- | (SEQ ID NO: 31) |
| P832_attB5 | |
| Tn5 ME B | GTCTCGTGGGCTCGGAGATGTGTATAAGAGACAGCTCACCATCAACTTTGTATACAAAAG |
| adaptor- | (SEQ ID NO: 32) |
| P859_attB5 | |
| Tn5 ME B | GTCTCGTGGGCTCGGAGATGTGTATAAGAGACAGCTCGGCAACTTTGTATACAAAAG |
| adaptor- | (SEQ ID NO: 33) |
| P887_attB5 | |

### Example 8: Design and Generation of Scarless Site Specific Mutations

Gateway® compatible fragments were designed to include the 5′ and 3′ pBac sequences required for transposition internal to the targeting homology arms (FIG. 4). Fragments were synthesized by Twist Biosciences and Gateway® cloned into pJAT10 (SEQ ID NO: 6) as described above.

### Example 9: Design and Generation of Targeted Inversions

Gateway® compatible fragments were designed with homology arms configured to promote chromosomal inversions (FIG. 5). Fragments were synthesized by Twist Biosciences and Gateway® cloned into pJAT21 (SEQ ID NO: 7) and pJAT25 (SEQ ID NO: 8) as described above. Both plasmids were injected simultaneously into flies carrying a nos-Cas9 transgene.

### Example 10: Example of Protocol for Designing Homology Arms and Cloning Steps

pJAT plasmids allow simple cloning of a gRNA and homology arms for CRISPR/Cas9 mediated homology directed repair. The gRNA is expressed at high levels from a U6-3 promoter and spliced out of the transcript by flanking native tRNA sequences. The homology arms are designed to integrate an attP site in-frame to a gene of interest. Subsequently phiC31-mediated attP/attB integration can be used to integrate T2A-Gal4, T2A-LexA, T2A-AD, or T2A-DBD in frame to the target gene.

To make plasmid DNA, the following steps can be taken conducted

1. Transform the supplied plasmid P777 into ccdB Survival cells following the standard protocol. Plate cells on LB_Ampicillin (100 μug/mL) and grow at 37° C. at least 15 hours.

2. Pick one colony and grow in 3 mL LB+antibiotics, e.g., Ampicillin, Chloramphenicol, or Spectinomycin) overnight, shaking at 37° C. Perform standard mini-prep next day. Antibiotic concentrations: Ampicillin: 100 μg/mL; Chloramphenicol: 25 μg/mL; Spectinomycin: 50 μg/mL.

To design and synthesize attL cloning arms, the following steps can be conducted.

3. Using computational tools (e.g., tools available at www.flyrnai.orq/crispr3/web/), identify a predicted high efficiency gRNA target site in the coding region of the gene of interest (target gene). Identify homology arms that precisely, or nearly precisely flank the expected Cas9 cut site, but that keep the attP site in frame. If it is intended that the attP site will be used to later integrate T2A-effector reagents, then ensure that the left homology arm ends at codon position 3 within the target gene.

4. Using sequence editing software (e.g., Geneious®), cut and paste the 20 gRNA target recognition sequence and the left and right homology arms into provided template sequences. Double check to make sure all the sequences have been inserted in the correct orientation. For example, the gRNA should be in forward direction relative target gRNA sequence, not necessarily in forward direction relative to the genomic target. Ensure that the attL3 and attL4 (and, similarly, the attL5 and attL6) are oriented inwards, towards each other. Replace the gRNA sequence indicated with your target 20 bp gRNA. Then concatenate: attL3+Homology Arm Left+attL4 and attL5+Homology Arm Right+attL6.

5. Synthesize the left and right homology arm pieces with the attL sites. For example, the tools available from Twist Bioscience® can be used to synthesize arms and leave their adaptor sequences on. This is cheaper than removing the adaptors and the adaptors are removed during Gateway® cloning. Sometimes the Twist QC step will flag the sequence as too complex to be sequenced. This usually results from regions containing high variance in GC content. Based on experience, on can edit these AT or GC rich regions in the homology arms with one or a few mutations to balance the GC content. DO NOT edit the attL sites or the tRNA-gRNA-tRNA region.

For cloning, the following steps can be conducted:

6. Perform Gateway® cloning reaction: 1 uL 100 ng/uL double Gateway® plasmid; 0.5 uL 50 ng/uL attL3-tRNA-gRNA-LeftHomologyArm-attL4 synthesized dsDNA; 0.5 uL 50 ng/uL attL5-RightHomologyArm-attL6 synthesized dsDNA; 0.25 uL LR Clonase II enzyme mix (Thermo Fisher). Incubate at least 12 hours at 25° C. in thermocycler with heated lid. Note: The published protocol specifies 1 hour incubation, but the double Gateway® reaction is less efficient than single Gateway® cloning, and a longer incubation has been found to improve cloning efficiency.

7. 0125 μL Proteinase-K (from Clonase kit), Incubate 37° C. 20 min in thermocycler with heated lid.

8. Gently mix the entire 2.3 μL reaction with 50 μL of DH5alpha (or equivalent) competent cells. Mix & Go competent cells (Zymo Research) has been found to provide higher cloning efficiency than other leading brands and are considerably cheaper than other brands. (For further cost savings, the supplied 50 μL aliquots can be split into two 25 μL aliquots by gently pipetting 25 uL into a new tube on ice.) Hundreds of colonies are routinely observed on plates. Very occasionally only a few colonies are seen, but they are usually correct.

9. Let mix site on ice for 30 min.

10. Gently, but rapidly move tube containing cells to 42° C. block or water bath for 30 see without shaking.

11. Move tube to ice for a few minutes.

12. Add 150 μL SOC

13. Tape tube on its side to base of a 37° C. shaking incubator and shake at 300 RPM for 1 hour

14. Plate 100 μL on Agar plates containing Tryptone-Yeast extract+Spectinomycin+Sucrose and grow overnight at 37° C.

To make Tryptone-Yeast+Sucrose plates, the following steps are conducted.
a) Make the agar solution: 10 g tryptone; 5 g yeast extract; 15 g agar (or whatever amount typically used per liter); 750 ml water
b) Autoclave
c) Separately make the sucrose solution: 100 g sucrose; 250 ml water
d) Filter sterilize using a 0.2 micron filter.
e) When autoclaved agar is cooled, mix the sucrose solution into the agar, add appropriate volume of antibiotics (e.g., Spectinomycin to 100 μg/mL) and pour plates.

15. Pick colonies into LB+Spectinomycin. Grow overnight at 37° C. with shaking and perform miniprep. Check that gRNA and homology arms are correctly integrated by sequencing plasmid. (Either whole plasmid sequencing can be performed, or just the ends of the integrated arms can be sequenced with SP6 and T7 primers.)

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Joung, J. K., and Sander, J. D. (2013). TALENs: a widely applicable technology for targeted genome editing. Nat. Rev. Mol. Cell Biol. 14, 49-55. 10.1038/nrm3486.
2. Beumer, K. J., Trautman, J. K., Bozas, A., Liu, J. L., Rutter, J., Gall, J. G., and Carroll, D. (2008). Efficient gene targeting in Drosophila by direct embryo injection with zinc-finger nucleases. Proc Natl Acad Sci U A 105, 19821-19826. 10.1073/pnas.0810475105.
3. Gratz, S. J., Ukken, F. P., Rubinstein, C. D., Thiede, G., Donohue, L. K., Cummings, A. M., and Oconnor-Giles, K. M. (2014). Highly specific and efficient CRISPR/Cas9-catalyzed homology-directed repair in Drosophila. Genetics 196, 961-971. 10.1534/genetics.113.160713.
4. Afif, H., Allah, N., Couturier, M., and Melderen, L. V. (2001). The ratio between CcdA and CcdB modulates the transcriptional repression of the ccd poison-antidote system. Mol. Microbiol. 41, 73-82. https://doi.org/10.1046/j.1365-2958.2001.02492.x.
5. Port, F., and Bullock, S. L. (2016). Augmenting CRISPR applications in Drosophila with tRNA-flanked sgRNAs. Nat. Methods 13, 852-854. 10.1038/nmeth.3972.
6. Ding, Y., Berrocal, A., Morita, T., Longden, K. D. K. D., and Stem, D. L. D. L. (2016). Natural courtship song variation caused by an intronic retroelement in an ion channel gene. Nature 536, 329-332. 10.1038/nature19093.
7. Kanca, O., Zirin, J., Hu, Y., Tepe, B., Dutta, D., Lin, W.-W., Ma, L., Ge, M., Zuo, Z., Liu, L.-P., et al. (2022). An expanded toolkit for Drosophila gene tagging using synthesized homology donor constructs for CRISPR-mediated homologous recombination. eLife 11, e76077. 10.7554/eLife.76077.
8. Aboulela, M., Tanaka, Y., Nishimura, K., Mano, S., Kimura, T., and Nakagawa, T. (2017). A dual-site gateway cloning system for simultaneous cloning of two genes for plant transformation. Plasmid 92, 1-11. 10.1016/j.plasmid.2017.05.001.
9. Li, X., Thomason, L. C., Sawitzke, J. A., Costantino, N., and Court, D. L. (2013). Positive and negative selection using the tetA-sacB cassette: recombineering and P1 transduction in Escherichia coli. Nucleic Acids Res. 41, e204. 10.1093/nar/gkt1075.
10. Stern, D. L. (2022). Transgenic tools for targeted chromosome rearrangements allow construction of balancer chromosomes in non-Melanogaster Drosophila species. G3 GenesGenomesGenetics 12, jkac030. 10.1093/g3journal/jkac030.
11. Stern, D. L. (2016). Tagmentation-Based Mapping (TagMap) of Mobile DNA Genomic Insertion Sites. bioRxiv, 1-7. 10.1101/037762.
12. Kanca, O., Zirin, J., Garcia-Marques, J., Knight, S. M., Yang-Zhou, D., Amador, G., Chung, H., Zuo, Z., Ma, L., He, Y., et al. (2019). An efficient CRISPR-based strategy to insert small and large fragments of DNA using short homology arms. eLife 8, e51539. 10.7554/eLife.51539.
13. Xiang, G., Zhang, X., An, C., Cheng, C., and Wang, H. (2017). Temperature effect on CRISPR-Cas9 mediated genome editing. J. Genet. Genomics 44, 199-205. 10.1016/j.jgg.2017.03.004.
14. Lamb, A. M., Walker, E. A., and Wittkopp, P. J. (2016). Tools and strategies for scarless allele replacement in Drosophila using CRISPR/Cas9. Fly (Austin) 0, 1-12. 10.1080/19336934.2016.1220463.
15. Nyberg, K. G., and Carthew, R. W. (2022). CRISPR-/Cas9-Mediated Precise and Efficient Genome Editing in Drosophila. Methods Mol. Biol. Clifton NJ 2540, 135. 10.1007/978-1-0716-2541-5_6.
16. Miller, D. E., Cook, K. R., and Hawley, R. S. (2019). The joy of balancers. PLoS Genet. 15, e1008421. 10.1371/journal.pgen.1008421.
17. Groth, A. C., Fish, M., Nusse, R., and Calos, M. P. (2004). Construction of Transgenic Drosophila by Using the Site-Specific Integrase From Phage {phi}C31. Genetics 166, 1775-1782.
18. Stern, D. L., Crocker, J., Ding, Y., Frankel, N., Kappes, G., Kim, E., Kuzmickas, R., Lemire, A., Mast, J. D., and Picard, S. (2017). Genetic and Transgenic Reagents for Drosophila simulans, D. mauritiana, D. yakuba, D. santomea, and D. virilis. G3, 1-10. 10.1534/g3.116.038885/-/DC1.1.
19. Bai, H., Liu, L., An, K., Lu, X., Harrison, M., Zhao, Y., Yan, R., Lu, Z., Li, S., Lin, S., et al. (2020). CRISPR/Cas9-mediated precise genome modification by a long ssDNA template in zebrafish. BMC Genomics 21, 67. 10.1186/s12864-020-6493-4.
20. Reyrat, J.-M., Pelicic, V., Gicquel, B., and Rappuoli, R. (1998). Counterselectable Markers: Untapped Tools for Bacterial Genetics and Pathogenesis. Infect. Immun. 66, 4011-4017. 10.1128/IAI.66.9.4011-4017.1998.
21. Dobzhansky, T., and Sturtevant, A. H. (1937). Inversions in the chromosomes of Drosophila pseudoobscura. Genetics 23, 28-64.
22. Joron, M., Frezal, L., Jones, R. T., Chamberlain, N. L., Lee, S. F., Haag, C. R., Whibley, A., Becuwe, M., Baxter, S. W., Ferguson, L., et al. (2011). Chromosomal rearrangements maintain a polymorphic supergene controlling butterfly mimicry. Nature 477, 203-206. 10.1038/nature10341.
23. Engler, C., Kandzia, R., and Marillonnet, S. (2008). A One Pot, One Step, Precision Cloning Method with High Throughput Capability. PLoS ONE 3, e3647. 10.1371/journal.pone.0003647.
24. Ma, H., Wu, Y., Dang, Y, Choi, J-G, Zhang, J., and Wu, H. (2014). Pol III Promoters to Express Small RNAs: Delineation of Transcription Initiation. Mol Ther Nucleic Acids; 3(5): e161. 10.1038/mtna.2014.12.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A cloning vector, comprising: a cassette having a nucleotide sequence comprising a negative selection marker that is a ccdB gene, which is flanked by non-identical attR recombination recognition sequences.

2. The vector of claim 1, and further comprising a second cassette having a nucleotide sequence comprising a second negative selection marker that is not a ccdB gene, which is flanked by second non-identical attR recombination recognition sequences, wherein the non-identical attR recombination recognition sequences recited in claim 1 are distinct from the second non-identical attR recombination recognition sequences.

3. The vector of claim 1, and further comprising a multiple cloning site or donor DNA that is intended to be inserted into the genome by homology directed repair located between the cassettes.

4. The vector of claim 1, wherein the second negative selection marker is a sacB gene.

5. The vector of claim 1, and further comprising an additional selection marker.

6. The vector of claim 5, wherein the additional selection marker is an antibiotic resistance marker.

7. The vector of claim 1, and further comprising a first antibiotic resistance marker in the first cassette, a second antibiotic resistance marker in the second cassette, and a third antibiotic resistance marker outside the first and second cassettes.

8. The vector of claim 1, and further comprising a promoter outside the cassettes.

9. The vector of claim 1, wherein the donor DNA comprises:
a sequence providing a germline source or ubiquitously expressed source of an enzyme for creating double-strand breaks,
attP landing sites,
marker genes, or
a split-fluorescent protein system to report on simultaneous homology-directed repair (HDR).

10. A donor vector, comprising:
a homology arm sequence flanked by non-identical attR recombination recognition sequences; and
a short guide RNA (sgRNA) sequence adjacent the homology arm sequence and separated from the homology arm by an RNA polymerase termination sequence.

11. The vector of claim 10, and further comprising a second homology arm sequence flanked by second non-identical attR recombination recognition sequences, wherein the first non-identical attR recombination recognition sequences are distinct from the second non-identical attR recombination recognition sequences.

12. The vector of claim 10, and further comprising a pair of tRNA sequences flanking the sgRNA to provide precise splicing of sgRNA products from the RNA transcript; a donor DNA between the homology arms; and a transfer RNA (tRNA) sequence is operably linked to the gRNA sequence.

13. The vector of claim 10, wherein tRNA sequences flank and are operably linked to the gRNA sequence.

14. The vector of claim 13, wherein the gRNA is a single-guide RNA (sgRNA), comprising a CRISPR RNA (crRNA) sequence and a trans-activating CRISPR RNA (tracrRNA) sequence.

15. The vector of claim 10, comprising a gRNA region that comprises a first tRNA sequence, a crRNA sequence, a tracrRNA sequence, and a second tRNA sequence.

16. The vector of claim 10, wherein the donor DNA/payload comprises:
a sequence providing a germline source of an enzyme for creating double-strand breaks,
attP landing sites,
marker genes, or
a split-fluorescent protein system to report on simultaneous homology-directed repair (HDR).

17. A method of preparing a donor vector by simultaneously introducing two DNA fragments of interest into a cloning vector, comprising:
(a) providing the cloning vector according to claim 1;
(b) incubating the cloning vector with a first DNA fragment of interest flanked by attL recombination sites, a second DNA fragment of interest flanked by attL recombination sites, and a recombination enzyme; and
(c) selecting a vector in which the first DNA fragment of interest has replaced the first cassette, and the second DNA fragment of interest has replaced the second cassette, thereby obtaining the donor vector.

18. The method of claim 17, wherein the first DNA fragment of interest comprises a first homology arm sequence and a gRNA region sequence, and the second DNA fragment of interest comprises a second homology arm sequence.

19. A method of integrating a donor DNA into a genome of a cell, comprising:
(a) introducing the donor vector according to claim 17 into a cell; and
(b) providing to the cell a source of an enzyme for creating double-strand breaks (DSBs) guided by the sgRNA encoded in the donor vector.

20. The method of claim 19, and further comprising the step of confirming an efficiency of integration of greater than about 5, 10, 15, 20, or 25%.
